(19) United States
(12) Patent Application Publication
(10) Pub. No.: US 2010/0035855 A1
(43) Pub. Date: Feb. 11, 2010

(54) Method for treating or preventing a cardiovascular disease or condition utilizing estrogen receptor modulators based on APOE allelic profile of a mammalian subject

(75) Inventors: Roderick A. Hyde, Redmond, WA (US); Muriel Y. Ishikawa, Livermore, CA (US); Eric C. Leuthardt, St. Louis, MO (US); Dennis J. Rivet, Portsmouth, VA (US); Elizabeth A. Sweeney, Seattle, WA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/584,046

(22) Filed: Aug. 28, 2009

Related U.S. Application Data
(63) Continuation of application No. 12/455,272, filed on May 29, 2009.
Application No. 12/455,272 is a continuation-in-part of application No. 12/220,708, filed on Jul. 24, 2008.
Application No. 12/220,708 is a continuation-in-part of application No. 12/220,704, filed on Jul. 24, 2008.
Application No. 12/220,704 is a continuation-in-part of application No. 12/220,707, filed on Jul. 24, 2008.

(51) Int. Cl. A61K 31/56

(52) U.S. Cl. 514/182

(57) ABSTRACT

A method for treating cardiovascular disease in a mammalian subject includes providing to the subject at least one treatment regimen including at least one estrogen receptor modulator, wherein the at least one treatment regimen is determined based on the APOE allelic profile in the subject, and based on the steroid hormone levels prior to disease diagnosis and on current steroid hormone levels in the subject.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of U.S. patent application Ser. No. 12/220,708, entitled METHOD, DEVICE, AND MIT FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/220,704, entitled METHOD, DEVICE, AND KIT FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/220,707, entitled SYSTEM AND DEVICE FOR MAINTAINING PHYSIOLOGICAL LEVELS OF STEROID HORMONE IN A SUBJECT, naming Roderick A. Hyde, Muriel Y. Ishikawa, Dennis J. Rivet, Elizabeth A. Sweeney, Lowell L. Wood, Jr. and Victoria Y. H. Wood as inventors, filed 24 Jul. 2008, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

### SUMMARY

A method is described herein for treating or preventing a cardiovascular disease or condition in a mammalian subject or reducing the incidence of a cardiovascular disease or condition or alleviating the symptoms thereof. The method includes providing at least one estrogen receptor modulator as part of a treatment regimen that can be varied depending upon the APOE allelic profile of the mammalian subject. The method for treating cardiovascular disease in a mammalian subject includes providing at least one estrogen receptor modulator to the mammalian subject, wherein the at least one estrogen receptor modulator is determined based on the APOE allelic profile in the mammalian subject. The mammalian subject can have at least one reduced steroid hormone level compared to a steroid hormone level prior to disease diagnosis. In a female subject, the at least one treatment regimen can be determined based on the premenopausal cyclic steroid hormone levels in the subject and on current perimenopausal or menopausal cyclic steroid hormone levels in the subject. In an aspect, the subject's APOE allelic profile is ε2/ε3 positive. In a further aspect, the at least one estrogen receptor modulator includes at least one selective estrogen receptor α agonist. In an aspect, the subject's APOE allelic profile is ε4 positive. In a further aspect, the at least one treatment regimen including at least one selective estrogen receptor β agonist.

The method can further include providing at least one treatment regimen to the mammalian subject including at least one replacement therapy for one or more steroid hormones or metabolites or modulators thereof, wherein the at least one replacement therapy is configured to increase at least one steroid hormone level, e.g., levels of an estrogen and/or a progestogen, in the subject. The method includes providing the treatment regimen configured to maintain a substantially physiological level of one or more steroid hormones in a mammalian subject in need thereof. The method further includes providing the treatment regimen configured to maintain a substantially physiological cyclic pre-menopausal level of one or more steroid hormones in a female mammalian subject in need thereof.

A method described herein for treating a cardiovascular disease or condition in a mammalian subject comprises providing to the mammalian subject at least one estrogen receptor modulator, wherein the at least one estrogen receptor modulator is determined based on an APOE allelic profile in the mammalian subject, and wherein the mammalian subject has at least one reduced steroid hormone level compared to a steroid hormone level prior to disease diagnosis. The subject's APOE allelic profile can be at least one of APOE ε2 positive and APOE ε3 positive. The at least one estrogen receptor modulator can include at least one selective estrogen receptor α agonist. In an aspect, the subject's APOE allelic profile is ε4 negative. The at least one selective estrogen receptor α agonist can include, but is not limited to, 17β-estradiol, propylpyrazole triol, or 3,17-dihydroxy-19-nor-17α-pregna-1,3,5 (10)triene-21,16α-lactone. The subject's APOE allelic profile can be ε4 positive. The at least one estrogen receptor modulator can include at least one selective estrogen receptor β agonist. The at least one selective estrogen receptor β agonist can include, but is not limited to, diarylpropionitrile, ERB-041, WAY-202196, WAY-214156, or (2,8-dihydroxy-6H-dibenzo[c,h]chromene-4,12-dicarbonitrile), 8-vinylestra-1,3,5 (10)-triene-3,17β-diol. The at least one estrogen receptor modulator can be configured to maintain the subject's one or more steroid hormones or metabolites or modulators thereof at substantially physiological pre-disease cyclic levels, and the at least one estrogen receptor modulator can be in an amount effective to reduce the cardiovascular disease or condition or alleviate symptoms thereof in the subject. In an aspect, the mammalian subject is female. In a further aspect, the mammalian subject is perimenopausal or postmenopausal. The at least one estrogen receptor modulator can be determined at least in part based on the premenopausal cyclic steroid hormone levels in the mammalian subject and on current cyclic steroid hormone levels in the mammalian subject. In an aspect, n the mammalian subject is male. The cardiovascular disease or condition can include, but is not limited to, at least one of heart disease, bleeding, inflammation, atherosclerosis, hypercholesterolemia, acute myocardial infarction, myocardial ischemia, reperfusion injury, venous thrombosis, coronary insufficiency, coronary heart disease, valve disease, atherothrombotic stroke, or intermittent claudication.

The method described herein can further include providing at least one treatment regimen including at least one replacement therapy including one or more steroid hormones or metabolites or modulators thereof. The at least one treatment regimen including the least one replacement therapy can be configured to increase levels of one or more of an estrogen or a progestogen, or metabolites or modulators thereof. The at least one treatment regimen can include replacement therapy with one or more of an estrogen or a progestogen.

The method described herein can further include providing to the mammalian subject at least one treatment regimen including at least one aromatase inhibitor. The method can further include providing to the mammalian subject at least one treatment regimen including at least one or more of an estrogen receptor α antagonist or an estrogen receptor β antagonist. The method can further include providing to the mammalian subject at least one treatment regimen including one or more of an aromatase inhibitor, an estrogen receptor α antagonist or an estrogen receptor β antagonist. The at least one estrogen receptor modulator can be configured to maintain the subject's one or more steroid hormones or metabolites or modulators thereof at substantially physiological pre-disease levels. The current cyclic steroid hormone levels can be based on steroid hormone levels during a period of disease in the subject.

The method described herein can further include determining the one or more steroid hormones levels in the subject during a treatment period. The treatment period can include a time period preceding treatment with the at least one estrogen receptor modulator. The treatment period can include a time period during treatment with the at least one estrogen receptor modulator. The determining of the one or more steroid hormones levels can occur at multiple time points during the treatment period. The method can further include providing to the subject the at least one estrogen receptor modulator and at least one treatment regimen including at least one replacement therapy including one or more steroid hormones or metabolites or modulators thereof configured to maintain the subject's one or more steroid hormones or one or more metabolites or modulators thereof at substantially physiological levels. The at least one treatment regimen can include replacement therapy with one or more of an estrogen and a progestogen. The method can further include determining a genetic profile including at least an APOE profile of the subject. The at least one treatment regimen can be determined based at least in part on one or more of a time-history of serum steroid hormone levels in the subject, on inferred peak values or minimal values of serum steroid hormone levels in the subject, on age of the subject, or on categorization relative to profiles of patient populations. The at least one treatment regimen can be determined based at least in part on Fourier analysis of the cyclic steroid hormone levels in the subject, or on harmonic analysis of the cyclic steroid hormone levels in the subject. The at least one treatment regimen can be determined based at least in part on scaled values of the steroid hormone levels prior to the disease diagnosis in the subject. The at least one treatment regimen can be determined based at least in part on the scaled value approximately equal to one. The at least one treatment regimen can be determined based at least in part on the scaled value dependent on age of the subject.

A system described herein comprises a signal-bearing medium including one or more instructions for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition. The mammalian subject's APOE allelic profile can be at least one of APOE ε2 positive and APOE ε3 positive. The at least one estrogen receptor modulator can include at least one selective estrogen receptor α agonist. The mammalian subject's APOE allelic profile can be ε4 negative.

The system described herein can further include providing one or more instructions for determining the at least one treatment regimen including at least one replacement therapy for one or more steroid hormones or metabolites or modulators thereof. The at least one treatment regimen including the least one replacement therapy can be configured to increase levels of one or more of an estrogen and a progestogen, or metabolites or modulators thereof. The at least one selective estrogen receptor α agonist can include, but is not limited to, 17β-estradiol, propylpyrazole triol, or 3,17-dihydroxy-19-nor-17α-pregna-1,3,5 (10)triene-21,16α-lactone. The mammalian subject's APOE allelic profile can be ε4 positive. The at least one estrogen receptor modulator can include at least one selective estrogen receptor β agonist. The at least one treatment regimen includes the at least one replacement therapy can be configured to increase levels of one or more of an estrogen and a progestogen, or metabolites or modulators thereof. The at least one treatment regimen can include replacement therapy with one or more of an estrogen and a progestogen. The at least one selective estrogen receptor β agonist can include, but is not limited to, diarylpropionitrile, ERB-041, WAY-202196, WAY-214156 (2,8-dihydroxy-6H-dibenzo[c,h]chromene-4,12-dicarbonitrile), or 8-vinylestra-1,3,5 (10)-triene-3,17β-diol. The at least one estrogen receptor modulator can be configured to maintain the mammalian subject's one or more steroid hormones or metabolites or modulators thereof at substantially physiological cyclic levels, and the at least one estrogen receptor modulator can be in an amount effective to reduce cardiovascular disease or condition or alleviate symptoms thereof in the mammalian subject. The mammalian subject can be female. The female subject can be perimenopausal or postmenopausal. The at least one treatment regimen can be determined at least in part based on the premenopausal cyclic steroid hormone levels in the mammalian subject and on current cyclic steroid hormone levels in the mammalian subject. The mammalian subject can be male. The system can further include one or more instructions for inputting information associated with the steroid hormone levels prior to disease diagnosis and on current steroid hormone levels in the mammalian subject. The at least one treatment regimen can be configured to maintain a substantially physiological cyclic level of one or more steroid hormones prior to disease diagnosis in the mammalian subject in need thereof, and the at least one treatment regimen is in an amount effective to reduce cardiovascular disease or condition or alleviate symptoms thereof in the mammalian subject. The signal bearing medium can include a computer readable medium. The signal bearing medium can include a recordable medium. The signal bearing medium can include a communications medium.

A device described herein comprises a system including a signal-bearing medium including one or more instructions for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to disease diagnosis. The mammalian subject's APOE allelic profile can be at least one of APOE ε2 positive and APOE ε3 positive. The at least one estrogen receptor modulator can include at least one selective estrogen receptor α agonist. The mammalian subject's APOE allelic profile can be ε4 negative. The mammalian subject's APOE allelic profile can be ε4 positive. The at least one estrogen receptor modulator can include at least one selective estrogen receptor β agonist. The device can further include one or more instructions for determining the at least one treatment regimen including at least one replacement therapy for one or more steroid hormones or metabolites or modulators thereof. The at least one treatment regimen including the least one replacement therapy can be configured to increase levels of one or more of an estrogen and a progestogen or metabolites or modulators thereof. The at least one treatment regimen can include replacement therapy with one or more of an estrogen and a progestogen. The mammalian subject can be female. The female subject can be perimenopausal or postmenopausal. The at least one treatment regimen can be determined based on the premenopausal cyclic steroid hormone levels in the mammalian subject and on current cyclic steroid hormone levels in the mammalian subject. The mammalian subject can be male.

A system described herein comprises at least one computer program included on a computer-readable medium for use with at least one computer system wherein the computer program includes a plurality of instructions including one or more instructions for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition. The mammalian subject's APOE allelic profile can be at least one of APOE ε2 positive and APOE ε3 positive. The at least one estrogen receptor modulator can include at least one selective estrogen receptor α agonist. The mammalian subject's APOE allelic profile can be ε4 negative. The mammalian subject's APOE allelic profile can be ε4 positive. The at least one estrogen receptor modulator can include at least one selective estrogen receptor β agonist. The system can further include one or more instructions for determining the at least one treatment regimen including at least one replacement therapy for one or more steroid hormones or metabolites or modulators thereof. The at least one treatment regimen including the least one replacement therapy can be configured to increase levels of one or more of an estrogen and a progestogen or metabolites or modulators thereof. The at least one treatment regimen can include replacement therapy with one or more of an estrogen and a progestogen. The mammalian subject can be female. The female subject can be perimenopausal or postmenopausal. The at least one treatment regimen can be determined based on the premenopausal cyclic steroid hormone levels in the mammalian subject and on current cyclic steroid hormone levels in the mammalian subject. The mammalian subject can be male.

A system described herein comprises circuitry for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition. The mammalian subject's APOE allelic profile can be at least one of APOE ε2 positive and APOE ε3 positive. The at least one estrogen receptor modulator can include at least one selective estrogen receptor α agonist. The mammalian subject's APOE allelic profile can be ε4 negative. The mammalian subject's APOE allelic profile can be ε4 positive. The at least one estrogen receptor modulator can include at least one selective estrogen receptor β agonist. The system can further include one or more instructions for determining the at least one treatment regimen including at least one replacement therapy for one or more steroid hormones or metabolites or modulators thereof. The at least one treatment regimen including the least one replacement therapy can be configured to increase levels of one or more of an estrogen and a progestogen or metabolites or modulators thereof. The at least one treatment regimen can include replacement therapy with one or more of an estrogen and a progestogen. The mammalian subject can be female. The female subject can be perimenopausal or postmenopausal. The at least one treatment regimen can be determined based on the premenopausal cyclic steroid hormone levels in the mammalian subject and on current cyclic steroid hormone levels in the mammalian subject. The mammalian subject can be male. The system can further include circuitry for inputting information associated with the steroid hormone levels prior to disease diagnosis and on current steroid hormone levels in the mammalian subject. The at least one treatment regimen can be configured to maintain a substantially physiological cyclic level of one or more steroid hormones prior to disease diagnosis in the mammalian subject in need thereof.

A method described herein for treating a cardiovascular disease or condition in a perimenopausal or menopausal mammalian subject, the subject characterized as having one or both of APOE ε2 positive or APOE ε3 positive allelic profile, the method comprises administering an estrogen receptor-α agonist in an amount and for a time sufficient to increase expression of one or both of an APOE ε2 or APOE ε3 gene; and administering at least one replacement therapy including one or more steroid hormones or metabolites or modulators thereof in an amount and for a time sufficient to cause the mammalian subject's steroid hormone level to be substantially similar to the mammalian subject's premenopausal steroid hormone level. The subject's APOE allelic profile can be ε4 negative. The at least one treatment regimen including the least one replacement therapy can be configured to increase levels of one or more of an estrogen and a progestogen, or metabolites or modulators thereof.

The at least one treatment regimen can include replacement therapy with one or an estrogen and a progestogen. The at least one selective estrogen receptor α agonist can include, but is not limited to, 17β-estradiol, propylpyrazole triol, or 3,17-dihydroxy-19-nor-17α-pregna-1,3,5 (10)triene-21,16α-lactone. The at least one estrogen receptor modulator can be configured to maintain the subject's one or more steroid hormones or metabolites or modulators thereof at substantially physiological pre-disease cyclic levels, and the at least one estrogen receptor modulator can be in an amount effective to reduce the cardiovascular disease or condition or alleviate symptoms thereof in the subject. The mammalian subject can be female. The female subject can be perimenopausal or postmenopausal. The at least one estrogen receptor modulator can be determined at least in part based on the premenopausal cyclic steroid hormone levels in the mammalian subject and on current cyclic steroid hormone levels in the mammalian subject. The mammalian subject can be male. The cardiovascular disease or condition can include, but is not limited to, at least one of heart disease, bleeding, inflammation, atherosclerosis, hypercholesterolemia, acute myocardial infarction, myocardial ischemia, reperfusion injury, venous thrombosis, coronary insufficiency, coronary heart disease, valve disease, atherothrombotic stroke, or intermittent claudication. The method can further include providing to the mammalian subject at least one treatment regimen including at least one aromatase inhibitor. The method can further include providing to the mammalian subject at least one treatment regimen including at least one or more of an estrogen receptor α antagonist or an estrogen receptor β antagonist. The method can further include providing to the mammalian subject at least one treatment regimen including at least one of an aromatase inhibitor, an estrogen receptor α antagonist or an estrogen receptor β antagonist. The at least one estrogen receptor modulator can be configured to maintain the subject's one or more steroid hormones or metabolites or modulators thereof at substantially physiological pre-disease levels. The current cyclic steroid hormone levels can be based on steroid hormone levels during a period of disease in the subject. The method can further include determining the one or more steroid hormones levels in the subject during a treatment period. The treatment period can include a time period preceding treatment with the at least one estrogen receptor modulator. The treatment period can include a time period during treatment with the at least one estrogen receptor modulator. The determining of the one or more steroid hormones levels can occur at multiple time points during the treatment period. The method can further include determining a genetic profile including at least an APOE profile of the subject. The at least one treatment regimen can be determined based at least in part on one or more of a time-history of serum steroid hormone levels in the subject, on inferred peak values or minimal values of serum steroid hormone levels in the subject, on age of the subject, or on categorization relative to profiles of patient populations. The at least one treatment regimen can be determined based at least in part on Fourier analysis of the cyclic steroid hormone levels in the subject, or on harmonic analysis of the cyclic steroid hormone levels in the subject. The at least one treatment regimen can be determined based at least in part on scaled values of the steroid hormone levels prior to the disease diagnosis in the subject. The at least one treatment regimen can be determined based at least in part on the scaled value approximately equal to one. The at least one treatment regimen can be determined based at least in part on the scaled value dependent on age of the subject.

A method described herein for treating a cardiovascular disease or condition in a perimenopausal or menopausal mammalian subject, the subject characterized as having APOE ε4 positive allelic profile, the method comprises administering an estrogen receptor-β agonist in an amount and for a time sufficient to decrease expression of an APOE ε4 gene; and administering at least one replacement therapy including one or more steroid hormones or metabolites or modulators thereof in an amount and for a time sufficient to cause the mammalian subject's steroid hormone level to be substantially similar to the mammalian subject's premenopausal steroid hormone level.

A method described herein for preventing a cardiovascular disease or condition in a perimenopausal or menopausal mammalian subject, the subject characterized as having one or both of APOE ε2 positive or APOE ε3 positive allelic profile, the method comprises administering an estrogen receptor-α agonist in an amount and for a time sufficient to increase expression of one or both of the APOE β2 or APOE ε3 gene; and administering at least one replacement therapy including one or more steroid hormones or metabolites or modulators thereof in an amount and for a time sufficient to cause the mammalian subject's steroid hormone level to be substantially similar to the mammalian subject's premenopausal steroid hormone level.

A method described herein for preventing a cardiovascular disease or condition in a perimenopausal or menopausal mammalian subject, the subject characterized as having APOE ε4 positive allelic profile, the method comprises administering an estrogen receptor-β agonist in an amount and for a time sufficient to decrease expression of an APOE ε4 gene; and administering at least one replacement therapy including one or more steroid hormones or metabolites or modulators thereof in an amount and for a time sufficient to cause the mammalian subject's steroid hormone level to be substantially similar to the mammalian subject's premenopausal steroid hormone level.

A kit described herein comprises at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition; and instructions for administering the at least one treatment regimen and for monitoring the effectiveness of the at least one treatment regimen in the mammalian subject. The at least one treatment regimen can provide varying dosages on a periodic basis to maintain the subject's substantially physiological cyclic steroid hormone levels. The periodic basis can be daily, weekly, or every 28 days. The mammalian subject's APOE allelic profile can be at least one of APOE ε2 positive and APOE ε3 positive. The at least one estrogen receptor modulator can include at least one selective estrogen receptor α agonist. The mammalian subject's APOE allelic profile can be ε4 negative. The mammalian subject's APOE allelic profile can be ε4 positive. The at least one estrogen receptor modulator can include at least one selective estrogen receptor β agonist. The at least one treatment regimen can further include at least one replacement therapy for one or more steroid hormones or metabolites or modulators thereof. The at least one treatment regimen including the least one replacement therapy can be configured to increase levels of one or more of an estrogen and a progestogen or metabolites or modulators thereof. The at least one treatment regimen can include replacement therapy with one or more of an estrogen and a progestogen.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B depict a diagrammatic view of one aspect of an exemplary embodiment of a method for treating cardiovascular disease and maintaining a substantially physiological cyclic pre-menopausal level of one or more steroid hormones in a mammalian subject in need thereof.

FIG. 2 depicts a logic flowchart of a method for treating cardiovascular disease.

FIG. 3 depicts some aspects of a system that may serve as an illustrative environment for subject matter technologies.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application uses formal outline headings for clarity of presentation. However, it is to be understood that the outline headings are for presentation purposes, and that different types of subject matter may be discussed throughout the application (e.g., method(s) may be described under composition heading(s) and/or kit headings; and/or descriptions of single topics may span two or more topic headings). Hence, the use of the formal outline headings is not intended to be in any way limiting.

A method is described herein for treating or preventing a cardiovascular disease or condition in a mammalian subject or reducing the incidence of a cardiovascular disease or condition or alleviating the symptoms thereof. The method includes providing at least one estrogen receptor modulator as part of a treatment regimen that can be varied depending upon the APOE allelic profile of the mammalian subject. The method for treating cardiovascular disease or condition in a mammalian subject includes providing at least one estrogen receptor modulator to the mammalian subject, wherein the at least one estrogen receptor modulator is determined based on the APOE allelic profile in the mammalian subject. The mammalian subject can have at least one reduced steroid hormone level compared to a steroid hormone level prior to disease diagnosis. In a female subject, the at least one treatment regimen can be determined based on the premenopausal cyclic steroid hormone levels in the subject and on current perimenopausal or menopausal cyclic steroid hormone levels in the subject. In an aspect, the subject's APOE allelic profile is at least one of APOE ε2 positive and APOE ε3 positive. In a further aspect, the at least one estrogen receptor modulator includes at least one selective estrogen receptor α agonist. In an aspect, the subject's APOE allelic profile is ε4 positive. In a further aspect, the at least one treatment regimen including at least one selective estrogen receptor β agonist.

The method can further include providing at least one treatment regimen to the mammalian subject including at least one replacement therapy for one or more steroid hormones or metabolites or modulators thereof, wherein the at least one replacement therapy is configured to increase at least one steroid hormone level, e.g., levels of an estrogen and/or a progestogen, in the subject. The method includes providing the treatment regimen configured to maintain a substantially physiological level of one or more steroid hormones in a mammalian subject in need thereof. The method further includes providing the treatment regimen configured to maintain a substantially physiological cyclic pre-menopausal level of one or more steroid hormones in a female mammalian subject in need thereof.

In humans, there are three alleles of APOE (ε2, ε3, and ε4) and hence 6 different genotypes (ε2/ε2, ε2/ε3, ε2/ε4, ε3/ε3, ε3/ε4, and ε4/ε4). When the subject's APOE allelic profile is ε2 positive and/or ε3 positive (genotypes ε2/ε2, ε2/ε3, or ε3/ε3), the treatment regimen can include at least one estrogen receptor modulator, e.g., at least one selective estrogen receptor α agonist or selective estrogen receptor α antagonist. The treatment regimen including an estrogen receptor α agonist results in an increase in APOE ε2 gene expression and ApoE2 protein and/or APOE ε3 gene expression and ApoE3 protein in the mammalian subject having an ε2 positive and/or 3 positive allelic profile. In a further aspect, the subject's APOE allelic profile is ε4 negative. The method for treating cardiovascular disease or condition includes providing at least one estrogen receptor α agonist to a mammalian subject as part of at least one treatment regimen to the mammalian subject with an APOE ε2 and/or ε3 profile. The treatment regimen includes at least one estrogen receptor α agonist optionally in combination with at least one replacement therapy for one or more steroid hormones or metabolites or modulators thereof. The at least one replacement therapy can increase levels of one or more of an estrogen and a progestogen, or metabolites or modulators thereof, in the subject. The subject having an APOE ε2 positive and/or ε3 positive allelic profile will respond to treatment with the at least one selective estrogen receptor α agonist by exhibiting an increase in APOE ε2 and/or ε3 gene expression or ApoE2 and/or ApoE3 protein product, apolipoprotein E, in the mammalian subject. The treatment regimen can optionally include the at least one replacement therapy for one or more steroid hormones. The treatment regimen is configured to reduce the incidence or severity of a cardiovascular disease or condition, or to reduce the symptoms thereof in the mammalian subject having an APOE ε2 positive and/or ε3 positive allelic profile.

When the mammalian subject's APOE allelic profile is ε4 positive (genotypes ε2/ε4, ε3/ε, or ε4/ε4), the treatment regimen can include at least one estrogen receptor modulator, e.g., at least one selective estrogen receptor β agonist or selective estrogen receptor β antagonist. The treatment regimen including an estrogen receptor agonist results in a decrease in APOE ε4 gene expression and decreased levels of ApoE ε4 protein in the mammalian subject having an ε4 positive allelic profile. The method for treating cardiovascular disease or condition includes providing at least one estrogen receptor β agonist to a mammalian subject as part of at least one treatment regimen to the mammalian subject with an APOE ε4 profile. The treatment regimen includes at least one estrogen receptor β agonist optionally in combination with at least one replacement therapy for one or more steroid hormones or metabolites or modulators thereof. The at least one replacement therapy can increase levels of one or more of an estrogen and/or a progestogen, or metabolites or modulators thereof, in the subject. The mammalian subject having an APOE ε4 positive allelic profile and treated with the at least one selective estrogen receptor β agonist optionally in combination with the at least one replacement therapy for one or more steroid hormones, will experience a reduced incidence or severity of cardiovascular disease or condition, or reduced symptoms thereof.

Hormone replacement or supplemental therapy has been used for some time to relieve symptoms of menopause or to provide protection from disorders such as osteoporosis. However, early and more recent studies have offered evidence that treatment with exogenous hormones carries risks, and limits have been suggested for treatments, including those on dosages and formulations. While incorporating these limitations, current therapies are still designed based on population data, with discussions on the need for individualized treatment regimens limited to health status and disease state without regard for individual medical history data on hormonal levels (for example, see Notelovitz, General Medicine, 8: 84, 2006. The Biologic and Pharmacologic Principles for Age-Adjusted Long-term Estrogen Therapy). Such proposals still rely on population-based “normal” ranges for hormone levels. In fact, levels of steroid hormones can differ greatly among individuals, and can be greatly affected by multiple factors including race, environment, and genotypes (for examples see Ellison et al., Lancet 342: 433-434, 1993; Pinheiro et al., Cancer Epidemiology Biomarkers & Prevention 14: 2147-2153, 2005; Núñez-de la Mora et al., PLoS Med 4(5): e167 2007; Jasienka, et al., Cancer Epidemiology, Biomarkers and Prevention 15: 2131-2135, 2006; Small, et al., Human Reproduction 20(8): 2162-2167, 2005; and Sharp et al., Am J Epidemiol 160: 729-740, 2004; which are incorporated herein by reference). Thus, regimens designed using population-based levels in many cases may be inappropriate for a patient, providing too much, too little, or the wrong type(s) of steroid hormones, potentially resulting in ineffectual or even harmful outcomes. In addition, much attention is now focusing on hormone treatment in younger women, such as women transitioning into and through natural menopause or women with a loss of ovarian function due to surgery, exposure, or disease. Studies in humans and animals provide evidence that pre-menopausal exposure and/or higher lifetime exposure to hormones confers protection against neurological disease (see, e.g., McLay, et al., J. Neuropsychiatry Lin. Neurosci. 15:161-167, 2003; Suzuki et al. PNAS USA, 104: 6013-6018, 2007; Ryan, et al. Int. Psychogeriatr. 20:47-56, 2008; and Morrison, et al., J. Neurosci. 26:10332-10348, 2006; which are incorporated herein by reference) and cardiovascular disease (van der Schouw et al., Lancet 16:714-8 1996). Current therapy and clinical trials (e.g., The Kronos Early Estrogen Prevention Study (KEEPS)) are now focusing on treating women transitioning into menopause and early menopause with estrogens, alone or in combination, administered orally or transdermally (see, e.g., Clarkson, Menopause 14: 373-84, 2007; Harman, et al., Climacteric 8(1):3-12, 2005; Qiao, et al. in Gender Medicine. 5 Suppl. A, S46-S64, 2008, which are incorporated herein by reference).

The methods for treatment of cardiovascular disease or condition described herein provide an individualized treatment to maintain a substantially physiological cyclic level of one or more steroid hormones in a mammalian subject in need thereof. The individualized treatment regimen includes one or more estrogen receptor modulators and at least one replacement therapy including one or more steroid hormones, and/or metabolites or modulators thereof. The at least one treatment regimen can include a pharmaceutical composition of at least one estrogen receptor modulator and optionally includes one or more steroid hormones, or metabolites, modulators, mimetics or analogs thereof. The treatment regimen can be based upon a genetic history of the subject, including, but not limited to, an APOE allelic profile of the subject, e.g., ε2, ε3, and ε4, or a combination thereof, for example, ε2/ε2, ε2/ε3, ε2/ε4, ε3/ε3, ε3/ε4, or ε4/ε4. In addition, the treatment regimen can be based upon information derived from pre-menopausal hormone levels or pre-disease hormone levels in the subject. In this context, a physiological level of a hormone includes the level of hormone measured at a given time. A physiological premenopausal level can be a level of the hormone as measured at a point in time during premenopause in a female subject. A physiological pre-disease level can be a level of the hormone as measured at a point in time prior to occurrence of disease or prior to surgery to treat a disease in a female or male subject. A current physiological level can be the level of the hormone as measured just prior to determining a treatment regimen. The physiological levels of the one or more steroid hormones of the female subject can be provided by collected measurements or can be provided as part of the subject's medical history, and the physiological premenopausal levels can include cyclic and/or temporal, e.g., age-related or weight-related, variations. A treatment regimen can be determined based on the physiological premenstrual levels and the current physiological levels. The determined treatment regimen can, for example, include maintaining physiological pre-menopausal hormone levels throughout perimenopause, menopause and/or postmenopause by administration of at least one estrogen receptor modulator and further including one or more exogenous hormones, metabolites, modulators, or related compounds or analogs thereof, and can include continual, cyclical, or time-dependent administration.

Determining a physiological level of a hormone can be based upon recurrent measurements of pre-menopausal or pre-disease hormone levels in the subject that can be used to provide at least one treatment regimen to the subject including at least one estrogen receptor modulator and optionally including replacement therapy for one or more estrogen receptor modulators, one or more steroid hormones, and/or metabolites or modulators thereof. The physiological pre-menopausal hormone levels or physiological pre-disease hormone levels in the subject can be obtained from past medical history, e.g., information from a past medical history provided by the subject, or present medical evaluation, e.g., information from a genetic history, e.g., an APOE allelic profile, current measurements by assay for pre-menopausal hormone levels or pre-disease hormone levels, or a combination thereof.

Prior to determining a treatment regimen, additional information regarding the physiological status of the subject can be gathered and assessed. For example, information on the subject's own history or his or her family's history of diseases including cardiovascular disease or condition, and genetic information can be collected. The medical evaluation can include a genetic profile of the subject regarding genes, genetic mutations, or genetic polymorphisms that can indicate risk factors that affect disease and/or are related to steroid hormone levels, hormone receptors, modulators (e.g., agonists or antagonists, of steroid hormones or steroid hormone receptors), enzymes involved in steroidogenesis, metabolites, or analogs thereof, or factors causing genetic disease or a genetic predisposition to disease in the subject. The genetic profile of the subject can include an APOE allelic profile to determine the presence of APOE alleles, ε2, ε3, and ε4, or a combination thereof, in the subject. Medical evaluation regarding genetic profiling or genetic testing can be provided as a current determination of genetic risk factors, or as part of the subject's medical history. Genetic profiling or genetic testing can be used to design a treatment regimen and thus determine an optimal level individualized for the subject of the at least one estrogen receptor modulator and further including the one or more steroid hormones, steroid hormone receptors, metabolites, or modulators or analogs thereof, obtained during a pre-menopausal or pre-disease period from the subject. A physician can use the genetic profiling or genetic testing information to determine a genetic basis for needed treatment of cardiovascular disease or condition including at least one estrogen receptor modulator optionally in combination with one or more steroid hormones configured to maintain a substantially physiological cyclic level of one or more steroid hormones in a mammalian subject in need thereof.

Prior to determining a treatment regimen, additional information regarding diseases, including cardiovascular disease or condition, and possible therapeutic treatment contained in population databases can be gathered and assessed. The medical evaluation can include information in a population database on disease risks, available drugs and formulations, and documented population responses to drugs and formulations.

A system is described herein that comprises at least one computer program included on computer-readable medium for use with at least one computer system and wherein the computer program includes a plurality of instructions including one or more instructions for determining at least one treatment regimen including at least one estrogen receptor modulator, wherein the at least one treatment regimen is determined based on the APOE allelic profile in the subject, and based on the steroid hormone levels prior to disease diagnosis and on current steroid hormone levels in the subject. A device is described that incorporates the system programmed to determine at least one treatment regimen to treat cardiovascular disease or condition and to maintain physiological cyclic levels of one or more steroid hormones or metabolites thereof in a mammalian subject in need thereof.

The method described herein for treating cardiovascular disease or condition in a mammalian subject in need thereof (e.g., pre-menopausal level in a female subject) is based on a genetic profile, e.g., APOE allelic profile, in the subject and based upon transitional changes in the levels of the one or more steroid hormones in the subject. Such changes considered in combination with the APOE allelic profile in the subject indicate a need for a treatment regimen including at least one estrogen receptor modulator and optionally including replacement therapy for the one or more steroid hormones, or metabolites or modulators thereof, to offset a decrease in the level of the one or more steroid hormones in the mammalian subject. The change in levels of the one or more steroid hormones can occur as a result of perimenopause, menopause, or postmenopause resulting in decreased levels of steroid hormones in a female subject. The changes in levels of the one or more steroid hormones can occur as a result of surgery (e.g., oophorectomy, ovariectomy, or orchiectomy), damage (e.g., loss of ovary function due to radiation or chemical exposure), or disease, (e.g., hypothyroidism, hyperthyroidism, or cancer) in a female subject or a male subject. In a further aspect, the method can restore a balance of the one or more steroid hormones in a subject in need thereof and reduce the incidence of cardiovascular disease or condition, or reduce symptoms thereof in the subject. The method can include, but is not limited to, providing to a subject at least one treatment regimen including at least one estrogen receptor modulator, wherein the at least one treatment regimen is determined based on the APOE allelic profile in the subject, configured to maintain a substantially physiological cyclic pre-menopausal level of one or more steroid hormones (e.g., a pre-menopausal level in a female subject) that closely mimics naturally cyclical dosage in a female or male subject to treat cardiovascular disease or condition, or reduce symptoms thereof associated with reduced levels of steroid hormones in the subject.

The method can further comprise determining the one or more steroid hormones levels in the subject during a treatment period. In a further aspect, the method includes at least one second treatment regimen to the subject including at least one estrogen receptor modulator optionally in combination with one or more steroid hormones, or metabolites or modulators thereof, at substantially physiological cyclic pre-menopausal levels. The at least one treatment regimen can be determined based at least in part on a time-history of serum steroid hormone levels in the subject, on inferred peak values or minimal values of serum steroid hormone levels in the subject, on age of the subject, or on categorization relative to profiles of patient populations. The at least one treatment regimen can be determined based on an APOE allelic profile of the subject.

Cyclical serum levels of steroid hormones include the serum levels over a period of time such as a menstrual cycle or a 28-day cycle, including the changes in levels during that time. Optimum cyclical serum levels include the optimum changes in the levels during a period of time such as a menstrual cycle or a 28-day cycle.

The method described herein for treating cardiovascular disease or condition in a mammalian subject comprises providing to the subject at least one treatment regimen including at least one estrogen receptor modulator, and optionally including replacement therapy for the one or more steroid hormones or metabolites or modulators thereof. The at least one treatment regimen can be determined based on a genetic history, e.g., an APOE allelic profile, and based on steroid hormone levels prior to disease diagnosis in the subject and on current steroid hormone levels in the subject, wherein the at least one treatment regimen is configured to maintain the subject's one or more steroid hormones or metabolites or modulators thereof at substantially physiological pre-disease levels.

Treatment for a cardiovascular disease or condition in a mammalian subject in need thereof that includes at least one treatment regimen including a pharmaceutical composition of at least one estrogen receptor modulator and, optionally, includes one or more steroid hormones or metabolites, modulators, mimetics, or analogs thereof, wherein the at least one treatment regimen is determined based on the APOE allelic profile in the subject. The treatment is directed to maintaining a substantially physiological level of one or more steroid hormones or metabolites or modulators thereof in a male subject. The treatment further is directed to maintaining a substantially physiological cyclic pre-menopausal level of one or more steroid hormones or metabolites or modulators thereof in a female subject. At least one treatment regimen including at least one estrogen receptor modulator, and optionally including replacement therapy for the one or more steroid hormones or metabolites or modulators thereof includes an individualized treatment for a disease or condition and maintains a substantially physiological cyclic pre-menopausal level of the one or more steroid hormones in a mammalian subject in need thereof. The at least one treatment regimen including at least one estrogen receptor modulator, and further including replacement therapy that is a pharmaceutical composition of one or more of the compounds or compositions as described herein, including but not limited to, natural or synthetic compounds with estrogenic activity; synthetic steroidal compounds having estrogenic activity; synthetic non-steroidal compounds having estrogenic activity; plant-derived phytoestrogens having estrogenic activity; esters, conjugates or prodrugs of suitable estrogens; androgens; modulators, including but not limited to selective estrogen receptor modulators (SERMs) and modulators of metabolic and/or synthetic pathways such as enzyme regulators; and modulators of signaling pathways; progestogens; progesterones, progestins, or any natural or synthetic compounds having progestational activity; gonadotropin hormones; or analogs, metabolites, modulators, mimetics, hormone precursors, metabolite precursors, biosynthetic enzymes, DNA encoding biosynthetic enzymes. The selective estrogen receptor modulators can include, but are not limited to, at least one selective estrogen receptor α agonist or at least one selective estrogen receptor β agonist. The at least one selective estrogen receptor α agonist can include, but is not limited to, 17β-estradiol or propylpyrazole triol, 3,17-dihydroxy-19-nor-17α-pregna-1,3,5 (10)triene-21,16α-lactone. Proc. Natl. Acad. Sci. USA 101: 5129-5134, 2004, which is incorporated herein by reference. The at least one selective estrogen receptor β agonist can include, but is not limited to, diarylpropionitrile, ERB-041 [Harris et al., Endocrinology 144: 4241-4249, 2003], WAY-202196, WAY-214156 (2,8-dihydroxy-6H-dibenzo[c,h]chromene-4,12-dicarbonitrile), 8-vinylestra-1,3,5 (10)-triene-3,170-diol, or a selective estrogen receptor modulator. Cvoro et al., J. Immunol., 180: 630-636, 2008; Proc. Natl. Acad. Sci. USA 101: 5129-5134, 2004, which are incorporated herein by reference.

The compound or composition can further include analogs, peptide mimetics, DNA encoding polypeptides of interest, or small chemical molecular mimetics of the one or more selective estrogen receptor modulators, one or more steroid hormones, or metabolites or modulators. The treatment is directed to treat cardiovascular disease or condition and to maintain a substantially physiological cyclic pre-menopausal level of one or more steroid hormones or metabolites thereof in a female subject. The treatment further is directed to treat cardiovascular disease or condition and to maintain a substantially physiological level of one or more steroid hormones or metabolites thereof in a male subject.

The at least one treatment regimen can further include one or more aromatase inhibitors and/or one or more estrogen receptor inhibitors. Aromatase inhibitors block the synthesis of estrogen and lower endogenous levels of estrogen in the subject. Non-selective aromatase inhibitors can include, but are not limited to, Aminoglutethimide, Testolactone (Teslac®). Selective aromatase inhibitors can include, but are not limited to, Anastrozole (Arimidex®), Letrozole (Femara®), Exemestane (Aromasin®), Vorozole (Rivizor®), Formestane (Lentaron®), or Fadrozole (Afema®). Additional aromatase inhibitors can include, but are not limited to, 4-androstene-3,6,17-trione or 1,4,6-androstatrien-3,17-dione.

Modulators can include activators and inhibitors. Modulators can increase or decrease hormones or other intermediates or receptors in a manner that regulates or increase steroid hormone levels. The modulator can be a physiologic modulator or a synthetic modulator. Activators are agents that, e.g., bind to, stimulate, increase, open, activate, facilitate, enhance activation, sensitize or up regulate the activity of steroid hormones or steroid hormone receptors, e.g., agonists. Inhibitors are agents that, e.g., bind to, partially or totally block stimulation, decrease, prevent, delay activation, inactivate, desensitize, or down regulate the activity of a steroid hormone intermediate, a receptor, or a steroid hormone receptor, e.g., antagonists. Modulators include agents that alter the interaction of the steroid hormone with the steroid hormone receptor. Modulators include compounds that act as activators or inhibitors of steroid hormones or steroid hormone receptors, including, but not limited to, proteins, peptides, lipids, carbohydrates, polysaccharides, lipoproteins, or glycoproteins. Modulators include genetically modified versions of naturally-occurring steroid hormones or other steroid hormone receptor ligands, e.g., with altered activity, as well as naturally occurring and synthetic ligands, antagonists, agonists, small chemical molecules and the like.

A treatment regimen can include a therapeutic amount of at least one estrogen receptor modulator, and optionally include one or more steroid hormones, or metabolites, modulators, or analogs thereof in a pharmaceutical composition, wherein the at least one treatment regimen is determined based on the APOE allelic profile in the subject. The treatment regimen can further include the schedule of changes in the dosage of the pharmaceutical composition to treat cardiovascular disease or condition and to maintain a substantially physiological cyclical serum level individualized for the subject. Treating or treatment includes the administration of the one or more selective estrogen receptor modulators, the one or more steroid hormones, or metabolites, modulators, or analogs thereof, to prevent or delay the onset of the symptoms, complications, or biochemical indicia of a disease, alleviating the symptoms, or arresting or inhibiting further development of the disease, condition, or disorder, e.g., cardiovascular disease or condition. Treatment can be prophylactic to prevent or delay the onset of the disease, or to prevent the manifestation of clinical or subclinical symptoms thereof, or can provide therapeutic suppression or alleviation of symptoms after the manifestation of the disease.

A mammalian subject can include, for example, a human, a non-human primate, as well as experimental animals such as rabbits, rats, mice, sheep, dogs, cats, cows, and other animals. A mammalian subject can further include, for example, a pet, an experimental animal, livestock, a zoo animal, or an animal in the wild.

A treatment regimen including at least one estrogen receptor modulator, wherein the at least one treatment regimen is determined based on the APOE allelic profile in the subject, and optionally including one or more steroid hormones, or metabolites, modulators, or analogs thereof, can be continuous and uninterrupted, that indicates that there is no break in the treatment regimen during the treatment period. Continuous, uninterrupted administration of a combination indicates that the combination can be administered during the entire treatment period, e.g., at least once daily or on a continuous and uninterrupted basis. The treatment regimen is directed to treat cardiovascular disease or condition and to maintain a therapeutic level or a determined cyclic level of the one or more steroid hormones, or metabolites, modulators, or analogs thereof. The treatment regimen can be provided to the subject by transdermal, subcutaneous, parenteral or oral administration. It is expected that the treatment period for the treatment regimen including at least one estrogen receptor modulator, and optionally including one or more steroid hormones, or metabolites, modulators, or analogs thereof will be for at least 30 days, preferably 120 days, and most preferably as long term treatment, and possibly indefinitely. One of the reasons for administering one or more selective estrogen receptor modulators and one or more steroid hormones or metabolites thereof is to treat a disease or condition associated with a decrease or absence of the one or more steroid hormones in the subject. Treatment periods also can vary depending on the symptoms to be treated. Physician evaluation along with patient interaction can assist the determination of the duration of treatment. For the treatment of cardiovascular disease or condition, or reduction in symptoms thereof, the treatment period could last from a period of weeks, months, years, or indefinitely. The administration of the treatment regimen including at least one estrogen receptor modulator, and optionally including replacement therapy for one or more steroid hormones, or metabolites or modulators thereof to a subject may need to be adjusted. Adjustments in the treatment regimen can depend upon the individual's medical history and fluctuations in current levels of steroid hormones in the subject. Administration of the treatment regimen can be adjusted to achieve the desired effect during a treatment period. Administration of at least one estrogen receptor modulator optionally in combination with one or more steroid hormones can be short term treatments or treatments of a finite term that can be less than the 30 day treatment period. It is anticipated that a patient may miss, or forget to take, one or a few dosages during the course of a treatment regimen, however, such patient is still considered to be receiving continuous, uninterrupted administration.

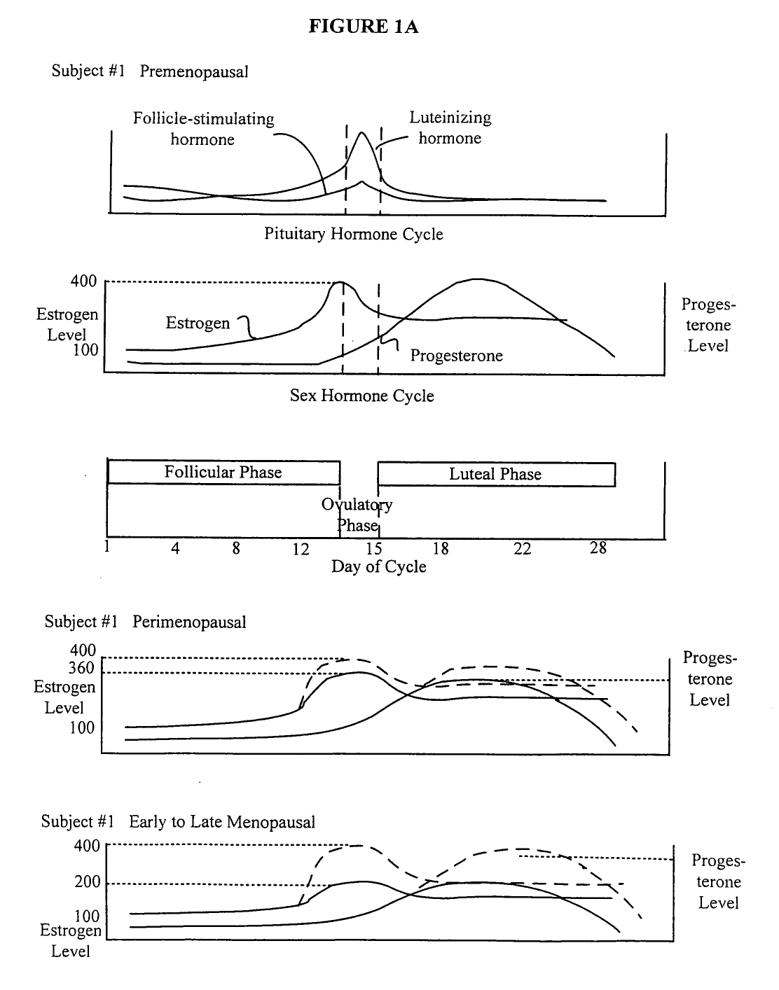

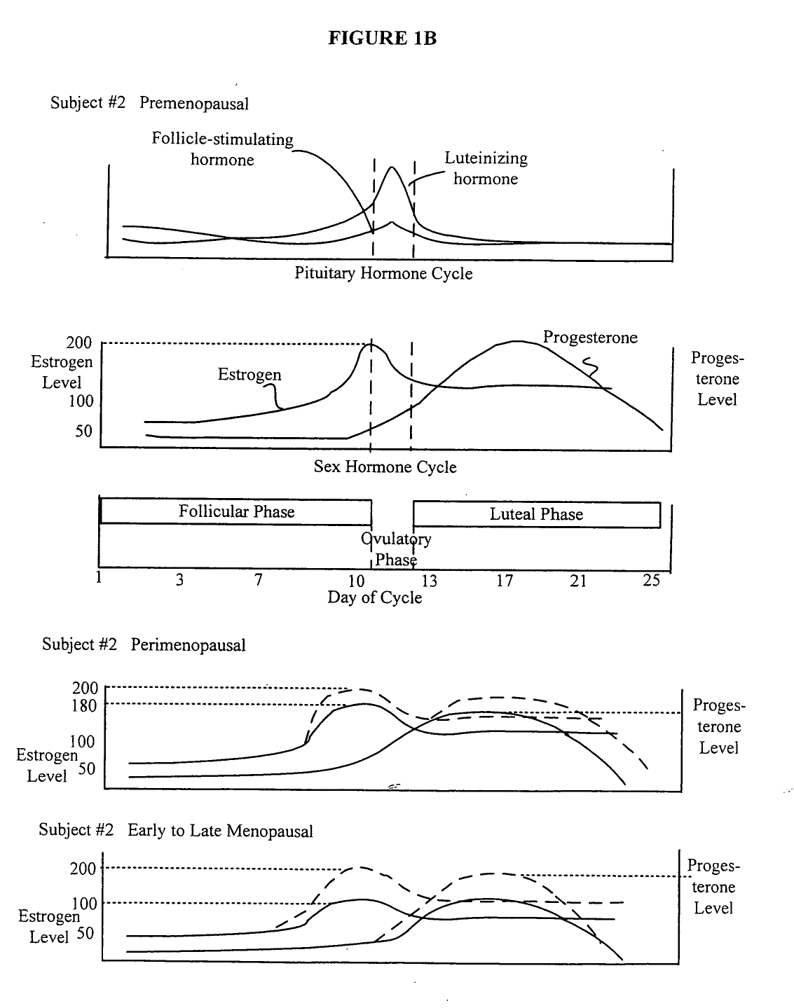

FIGS. 1A and 1B depict a diagrammatic view of an aspect of the methods and systems as described herein. The methods described herein for treating cardiovascular disease or condition in a mammalian subject in need thereof are individualized for a mammalian subject #1 (FIG. 1A) or for a mammalian subject #2 (FIG. 1B). Female subject #1 has premenopausal cyclic levels of steroid hormones, e.g., follicle stimulating hormone, luteinizing hormone, estrogen, and progesterone over a time period of 28 days. See solid lines on graph in FIG. 1A, Subject #1, Premenopausal. Female subject #1 in a perimenopausal condition has current cyclic levels of estrogen and progesterone reduced. Cyclic levels of estrogen and progesterone are further reduced in subject #1 in an early to late menopausal condition. See solid lines on graph in FIG. 1A; Subject #1, Perimenopausal. The method maintains a substantially physiological cyclic pre-menopausal level of one or more steroid hormones in the subject by providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject. In this case, the at least one treatment regimen including replacement therapy for the one or more steroid hormones or metabolites or modulators thereof is individualized and supplements the levels of estrogen and progesterone in the subject #1 to obtain pre-menopausal hormone levels. See dashed lines on graph in FIG. 1A, Subject #1, Perimenopausal or Early to Late Menopausal. Female subject #2 has premenopausal cyclic levels of steroid hormones, e.g., follicle stimulating hormone, luteinizing hormone, estrogen, and progesterone over a time period of 25 days. See solid lines on graph in FIG. 1B; Subject #2, Premenopausal. Female subject #2 has current cyclic levels of estrogen and progesterone reduced in a perimenopausal condition and current cyclic levels of estrogen and progesterone further reduced in an early to late menopausal condition. See solid lines on graph in FIG. 1B; Subject #2, Perimenopausal or Early to Late Menopausal. The method maintains a substantially physiological cyclic pre-menopausal level of one or more steroid hormones in the subject by providing to the subject at least one treatment regimen including replacement therapy for the one or more steroid hormones or metabolites or modulators thereof, wherein the at least one treatment regimen is determined based on pre-menopausal cyclic steroid hormone levels of the subject and on current cyclic steroid hormone levels of the subject. In this case, the at least one treatment regimen including replacement therapy for the one or more steroid hormones or metabolites or modulators thereof is individualized and supplements the levels of estrogen and progesterone in the subject #2 to obtain premenopausal hormone levels. See dashed lines on graph in FIG. 1B; Subject #2, Perimenopausal or Early to Late Menopausal.

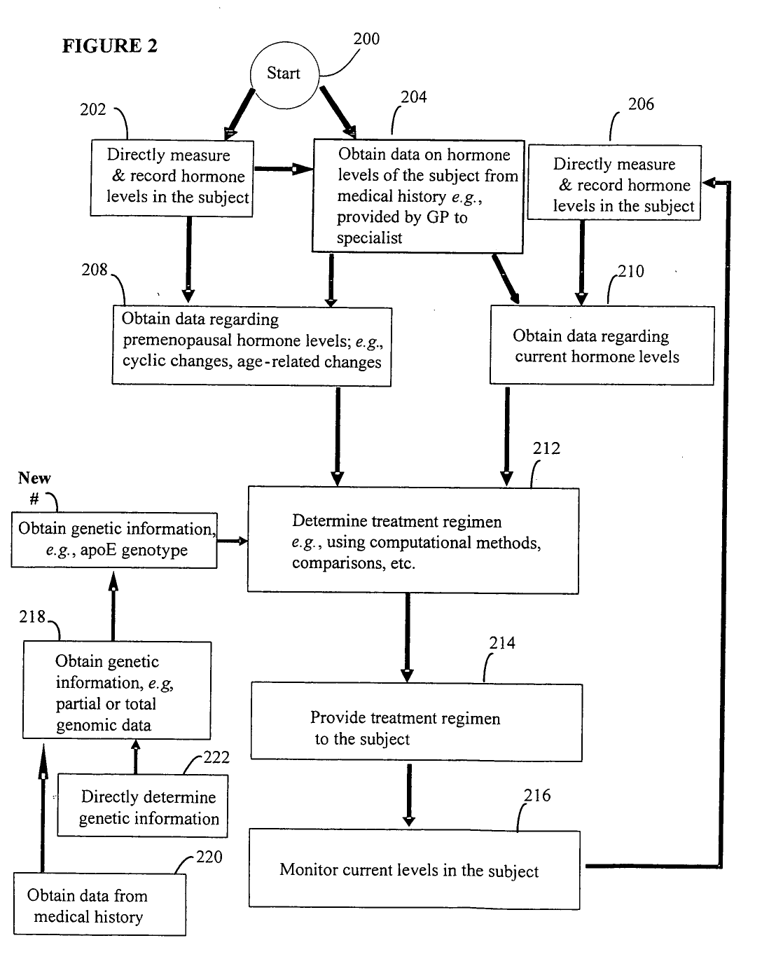

With reference to FIG. 2, depicted is a high-level logic flowchart of a process. Method step 200 shows the start of the process. Method step 202 depicts directly measuring and recording hormone levels in the subject. Method step 204 depicts obtaining data regarding hormone levels from a medical history of the subject. Method step 208 depicts obtaining data regarding premenopausal hormone levels in the subject from method steps 202 and/or 204. This data can reflect, e.g., cyclic hormonal changes or age-related hormonal changes in the subject. Method step 206 depicts directly measuring and recording hormone levels in the subject wherein the subject can be premenopausal, perimenopausal, early or late menopausal, or post menopausal. Method step 210 depicts obtaining data regarding current hormone levels from method steps 204 and/or 206. Method step 218 depicts obtaining genetic information, e.g., partial or total genomic data including APOE genotype, from a medical history 220 of the subject or from a direct determination of genetic information 222 from the subject. The genetic information, e.g., presence of APOE alleles ε2, ε3, or ε4, can be used to determine a treatment regimen. Method step 212 depicts determining a treatment regimen using methods e.g., including, but not limited to, computational methods or comparison methods. Method step 214 depicts providing at least one treatment regimen including replacement therapy for the one or more steroid hormones or metabolites or modulators thereof, to the subject. Method step 216 depicts monitoring current hormone levels during treatment of the subject. Method step 206 depicts directly measuring and recording hormone levels, e.g., during treatment of the subject. Method step 210 depicts obtaining data regarding current hormone levels. The data regarding current hormone levels is obtained from directly measuring and recording 206 current hormone levels during treatment of the subject and/or from obtaining data 204 on hormone levels from a medical history of the subject. The data is used to determine the proper treatment regimen 212 and alter or adjust the treatment regimen as needed, and providing the treatment regimen 214 to the subject. In an embodiment, method steps 202, 204, 206, 208, 210, 212, 214, 216, 218, 220, and/or 222 can include accepting input related to, for example, directly measuring and recording hormone levels in the subject, obtaining data on hormone levels from medical history of the subject, determining a treatment regimen, providing a treatment regimen and monitoring current hormone levels during treatment of the subject.

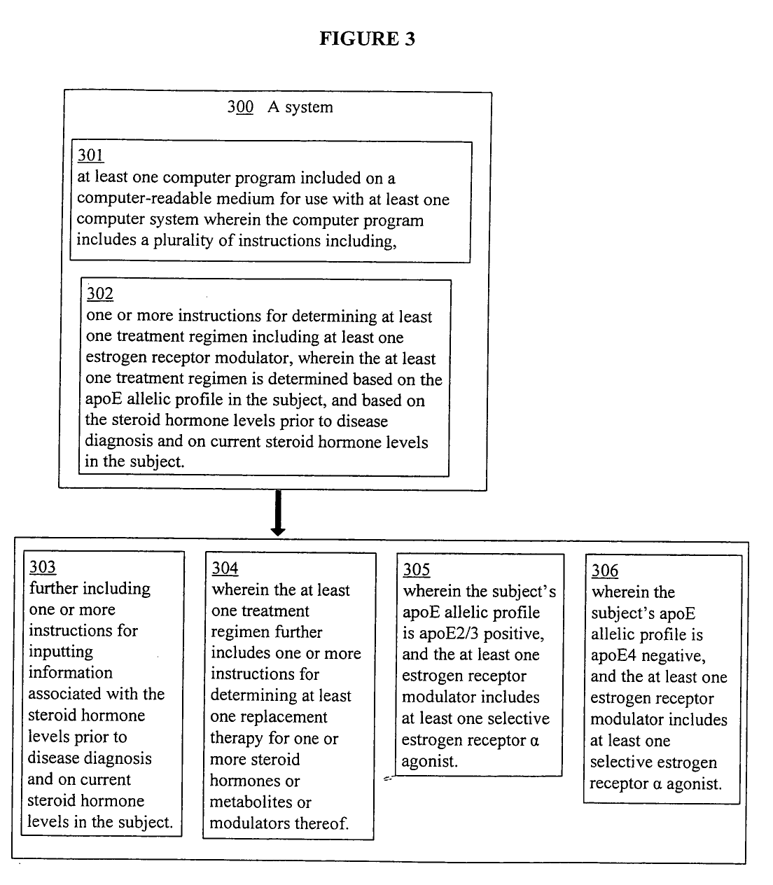

FIG. 3 depicts some aspects of a system that may serve as an illustrative environment for subject matter technologies. The system includes at least one computer program included on a computer-readable medium for use with at least one computer system wherein the computer program includes a plurality of instructions including one or more instructions for determining at least one treatment regimen including at least one estrogen receptor modulator, wherein the at least one treatment regimen is determined based on the apoE allelic profile in the subject, and based on the steroid hormone levels prior to disease diagnosis and on current steroid hormone levels in the subject.

### Methods for Treatment of Cardiovascular Disease or Condition Dependent Upon APOE Genotype in a Mammalian Subject

A method is described herein for treating a cardiovascular disease or condition in a subject that includes providing a treatment regimen that can be varied depending upon the APOE allelic profile of the subject. The method for treating cardiovascular disease or condition in a mammalian subject includes providing at least one estrogen receptor modulator to the subject, wherein the at least one estrogen receptor modulator is determined based on the APOE allelic profile in the subject. The treatment regimen can be further determined based on the steroid hormone levels prior to disease diagnosis and on current steroid hormone levels in the subject. The APOE allelic profile characterizes the APOE gene product, apolipoprotein E, in the mammalian subject. Apolipoprotein E (ApoE) is a liver polypeptide that has an important role in lipid metabolism by serving as a ligand for the LDL receptor. In humans, there are three alleles of APOE (ε2, ε3, and ε4) and hence 6 different genotypes ε2/ε2, ε2/ε3, ε2/ε4, ε3/ε3, ε3/ε4, and ε4/ε4). The gene for APOE is located on the long arm of chromosome 19 (19q13), where the three alleles ε2, ε3, and ε4 encode the six major APOE phenotypes. This functional polymorphism, which has several important effects on lipoprotein metabolism, increases serum total and low-density lipoprotein (LDL) cholesterol in the following order: ε2/ε2<ε3/ε2<ε4/ε2<ε3/ε3<ε4/ε3<ε4/ε4, in adults and children. APOE genotype distribution, in particular that of the APOE allele ε4, is associated with total and LDL cholesterol levels and also with cardiovascular morbidity. The frequencies of APOE genotypes vary in different age, sex, and race groups. APOE polymorphism is estimated to explain 4% to 15% of the variation in LDL cholesterol concentrations. In postmenopausal women, this variation has been reported to be greater than in premenopausal women. The response to cholesterol-lowering diet and statins has been shown to differ in subjects with different APOE genotypes. Postmenopausal hormone replacement therapy (HRT) changes serum lipoprotein concentrations favorably in APOE ε4-negative postmenopausal women compared to APOE ε4-positive postmenopausal women, which can explain about 25% to 50% of the cardioprotective effect of estrogen. Heikkinen, et al., Arterioscler Thromb Vasc Biol. 19: 402-407, 1999, which is incorporated herein by reference.

An APOE genotype in a female subject can modulate the effect of HRT on the development of atherosclerosis. Prospective studies have investigated the effect of long term HRT on the progression of atherosclerosis in postmenopausal women with or without the APOE ε4 phenotype, compared with controls of the same APOE ε4 status. The ε4 allele carrier status modulates the responses of lipoprotein metabolism to HRT. The effects of HRT on atherosclerosis progression in subjects with no ε4-allele, e.g., those carrying an ε2-, or ε3-allele, seems to be especially beneficial, compared with control treatment of subjects with same phenotype status but without HRT. These results help to understand, in more detail, the benefit and possible risk of HRT on atherosclerotic diseases dependent upon the APOE genotype of the subject. Lehtimaki, et al., J Clin Endocrinol Metab 87: 4147-4153, 2002, which is incorporated herein by reference.

Postmenopausal HRT has been shown to have favorable effects on the serum lipid profile in the female subject, and it also decreases the risk of cardiovascular disease or condition. The APOE genotype has influence on serum levels of lipids and lipoproteins; and APOE ε4 is associated with high total and LDL cholesterol levels. Genotype also influences the lipid responses to treatment with diet and statins. The effect of HRT in different APOE genotypes has been studied. In a population-based, prospective 5-year study, the effects of HRT were determined on the concentrations of serum lipids in APOE ε4-positive early postmenopausal women (genotypes ε3/ε4 and ε4/ε4) compared with those in APOE ε4-negative women (genotypes ε2/ε3 and ε3/ε3). The study found that, in postmenopausal Finnish women LDL, cholesterol levels in APOE ε4-negative subjects respond more favorably to HRT than those in APOE ε4-positive subjects. This finding has potential importance in postmenopausal women with hypercholesterolemia. Heikkinen, et al., Arterioscler Thromb Vasc Biol. 19: 402-407, 1999, which is incorporated herein by reference.

In some instances, information regarding a subject's risk for developing a cardiovascular disease may be unknown. Genetic profiling can be performed to determine relative risk. For example, a subject can undergo APOE genotyping for APOE ε2, ε 3, or ε4 alleles. A common method for APOE genotyping involves amplification by PCR of a 244 base-pair fragment within exon 4 of the APOE gene followed by digestion of the PCR fragment with the endonuclease HhaI, creating a characteristic pattern of DNA bands for each of the three common APOE alleles (ε2, ε3, or ε4) upon gel electrophoresis. See, e.g., Hegele. Clin Chem. 45: 1579-1580, 1999; Hixon, et al., J. Lipid Res. 31: 545-548, 1990; which are incorporated herein by reference. In particular, the APOE ε4 allele is strongly associated with cardiovascular disease in perimenopausal and postmenopausal women with the sensitivity ranging from 46 to 78 percent, and the specificity reaching nearly 100 percent.

A method for treating a cardiovascular disease or condition in a mammalian subject is described that provides at least one estrogen receptor modulator to the mammalian subject, wherein the at least one estrogen receptor modulator is determined based on the APOE allelic profile in the subject. The treatment regimen can further include at least one replacement therapy for one or more steroid hormones that can be determined based on the steroid hormone levels prior to disease diagnosis and on current steroid hormone levels in the subject. In an aspect, the subject's APOE allelic profile is at least one of APOE ε2 positive and APOE ε3 positive, and the at least one estrogen receptor modulator includes at least one selective estrogen receptor α agonist. In a further aspect, the subject's APOE allelic profile is ε4 positive, and the at least one treatment regimen including at least one selective estrogen receptor β agonist.

### Hormone Levels in a Subject

In a method for treating cardiovascular disease or condition in a mammalian subject in need thereof, the levels of estrogen receptors, estrogen receptor modulators, one or more steroid hormones, or metabolites or modulators thereof can be measured in one or more bodily fluids or tissues from the mammalian subject. Measurements of the levels of the one or more steroid hormones provide an individualized baseline for the substantially physiological cyclic pre-menopausal level in the subject and an indication of a need for the at least one treatment regimen including replacement therapy including at least one estrogen receptor modulator optionally in combination with replacement therapy for the one or more steroid hormones, or metabolites or modulators thereof. Examples of bodily fluids can include but are not limited to blood, serum, plasma, urine, urogenital secretions, sweat and or saliva. One or more steroid hormones or metabolites or modulators thereof that can be assayed in a bodily fluid or tissue can include but are not limited to estrogen fractions, for example, estrone [E1], estradiol (estradiol-17β, [E2]), and estriol [E3]; progesterone; androgens, for example, testosterone, dihydrotestosterone (DHT), dehydroepiandrosterone (DHEA), androstenedione, androst-5-ene-3β,17β-diol; non-sterol hormones, for example, follicle stimulating hormone, luteinizing hormone, inhibin B, anti-Mullerian hormone, and thyroid-related hormones; and modulators, for example metabolic precursors, metabolic enzymes, and hormone receptors, such as estrogen receptor α and estrogen receptor β. One or more steroid hormones, or metabolites, modulators, or analogs thereof can be measured in one or more bodily fluids or tissues, for example, by immunoassay, gas or liquid chromatography with or without mass spectrometry, or recombinant cell based assay. The one or more steroid hormones, or metabolites, modulators, or analogs thereof can also be measured, for example, by using sensor technology, including biosensors, protein arrays, and/or microfluidic devices, that can also be referred to as “lab-on-a-chip” systems.

For example, estrogen levels normally peak during the menstrual cycle at about day 15 during the follicular phase just prior to ovulation whereas progesterone levels peak at about day 25 in the luteal phase. Therefore, samples for hormone testing can be taken, for example, on multiple days over the course of one or more menstrual cycles. Because hormone levels can also fluctuate during the course of a 24 hour period, a specific time of day can be chosen for sample collection, for example, in early morning. In some instances, it can be beneficial to determine the hormonal fluctuations during the course of a 24 hour period. In this instance, sampling can be done multiple times during the course of a day and multiple days during the course of the menstrual cycle. In addition, hormone levels can undergo seasonal fluctuations, e.g., with higher levels recorded during the fall. Testing performed on a yearly or biennial basis can be performed at the same time during the year. In addition, since physiologic changes can influence hormone production, testing can be performed over several years and/or when a health index changes, e.g., a change in body-mass index. At that time, additional samples can be tested and/or other data can be gathered such as the subject's weight.

Levels of estrogen receptors, estrogen receptor modulators, steroid hormones, metabolites, or modulators thereof in a subject can be assayed in a bodily fluid or tissue using an immunoassay, for example, an enzyme-linked immunosorbent assay (ELISA; EIA) or a radioimmunoassay (RIA). In one type of an ELISA, the analysis is based on a competitive binding reaction to a specific antibody between an analyte, e.g., the steroid hormone, in the sample and a standard, e.g., a hormone standard, which is labeled with an enzyme. The antibody itself can be immobilized on a substrate such as a microtiter plate, tube, strip or beads, for example. The amount of labeled standard bound to the immobilized antibody is inversely proportional to the amount of analyte in the sample and can be determined by the addition of a chromogenic or fluorogenic substrate, that generates, upon interaction with the enzyme, a product detectable by an instrument such as a spectrophotometer or fluorometer. In another type of ELISA, the total amount of analyte bound to the immobilized antibody is detected by a secondary antibody (that can be the same antibody as the first antibody or different) labeled with an enzyme, and the assay developed by adding a chromogenic or fluorogenic substrate. In other types of assays, the analyte, standard hormone, or secondary antibody is labeled with a tag, for example a fluorescent tag, and is detected directly. Similarly, a chemiluminescent immunoassay can be used. Alternatively, the analyte, standard hormone, or secondary antibody can labeled with ¹²⁵Iodine for use in a radioimmunoassay. In these assays, quantification is determined by comparison to a standard curve generated using known amounts of analyte.

Antibodies or fragments thereof for use in an immunoassay can be generated against a hormone using standard methods, for example, such as those described by Harlow & Lane (Antibodies: A Laboratory Manual, Cold Spring Harbor Laboratory Press; 1^{st} edition 1988, which is incorporated herein by reference). Alternatively, an antibody fragment directed against a hormone can be generated using phage display technology (see, e.g., Kupper, et al. BMC Biotechnology 5:4, 2005, which is incorporated herein by reference). An antibody or fragment thereof could also be prepared using in silico design (Knappik et al., J. Mol. Biol. 296: 57-86, 2000, which is incorporated herein by reference). In addition or instead of an antibody, the assay can employ another type of recognition element, such as a receptor or ligand binding molecule. Such a recognition element can be a synthetic element like an artificial antibody or other mimetic. U.S. Pat. No. 6,255,461 (Artificial antibodies to corticosteroids prepared by molecular imprinting), U.S. Pat. No. 5,804,563 (Synthetic receptors, libraries and uses thereof), U.S. Pat. No. 6,797,522 (Synthetic receptors), U.S. U.S. Pat. No. 6,670,427 (Template-textured materials, methods for the production and use thereof), and U.S. Pat. No. 5,831,012, U.S. Patent Application 20040018508 (Surrogate antibodies and methods of preparation and use thereof); and Ye and Haupt, Anal Bioanal Chem. 378: 1887-1897, 2004; Peppas and Huang, Pharm Res. 19: 578-587 2002, provide examples of such synthetic elements and are incorporated herein by reference. In some instances, antibodies, recognition elements, or synthetic molecules that recognize a hormone is available from a commercial source, e.g., Affibody® affinity ligands (Abcam, Inc. Cambridge, Mass. 02139-1517; U.S. Pat. No. 5,831,012, incorporated here in by reference). For example, antibodies to estradiol, estrone, estriol, testosterone, DHEA, progesterone, follicle stimulating hormone, luteinizing hormone and estrogen receptors α and β are available from numerous commercial sources as listed in the Linscott's Directory of Immunological & Biological Reagents, Linscott's USA, 6 Grove St., Mill Valley, Calif. 94941 USA. Similarly, ELISA kits designed to measure one or more steroid hormones are commercially available. For example, ELISA kits for measuring estradiol, estrone, estriol, testosterone, DHEA, progesterone, follicle stimulating hormone, luteinizing hormone are commercially available from Cayman Chemical, Ann Arbor, Mich.; Calbiotech, Spring Valley, Calif.; Beckman Coulter, Fullerton, Calif. Other biomolecules can be developed to selectively bind to estrogen receptors, estrogen receptor modulators, steroid hormones or related molecules, modulators or metabolites, for example, DNA or RNA oligonucleotide based aptamers, and used in diagnostic assays (see, e.g., Jayasena. Clin. Chem. 45:1628-1650, 1999, which is incorporated herein by reference).

Alternatively, levels of one or more estrogen receptors, estrogen receptor modulators, steroid hormones, or modulators or metabolites thereof in a subject can be assayed in a bodily fluid or tissue using gas or liquid chromatography with or without mass spectrometry. For example, estradiol and estrone levels in human plasma can be simultaneously measured using a liquid chromatography-tandem mass spectrometry assay (see, e.g. Nelson, et al., Clin. Chem. 50:373-384, 2004, which is incorporated herein by reference). In this aspect, the serum samples are derivatized with dansyl chloride to increase the sensitivity of the assay and efficiency of ionization and separated from other components of the serum by liquid chromatography. Further purification and detection is done using mass spectrometry to differentiate between various steroid hormones. A more rapid method for detecting steroid hormones such as estradiol, estrone, estriol, 16-hydroxyestrone, and aldosterone, for example, using liquid chromatography, electrospray ionization and mass spectrometry (LC-ESI-MS/MS) has been described (see, e.g., Guo, et al., Clin. Biochem. 41:736-741, 2008, which is incorporated herein by reference). In this instance, the serum samples are deproteinized by extraction with acetonitrile followed by centrifugation at 13,000 rpm for 10 minutes. The supernatant is then loaded directly into the LC-ESI-MS/MS system where the samples are chromatographed. Standards are used to determine the elution profile of each steroid hormone, and the respective peaks are submitted to electrospray ionization followed by mass spectrometry. Known quantities of a given hormone are subjected to the same process and used to generate a standard curve against which the measured levels of hormone in the serum sample are compared.

Levels of one or more estrogen receptors, estrogen receptor modulators, steroid hormones, or modulators or metabolites thereof in a subject can also be assayed in a bodily fluid or tissue using a recombinant cell based assay or biosensor. In one instance, a yeast strain or a mammalian cell line, for example, is modified to express a recombinant hormone receptor that emits a measurable readout in response to binding an analyte, such as a steroid hormone. Klein, et al., describe development of a bioassay in Saccharomyces cerevisiae that have been transformed with the human estrogen receptor and an estrogen response element (ERE) upstream of the yeast iso-1-cytochrome C promoter fused to the structural gene for β-galactosidase (Klein, et al., J. Clin. Endocrinol. Metab. 80:2658-2660, 1995, which is incorporated herein by reference). Increased β-galactosidase activity in response to the presence of estrogen is assessed using calorimetric detection. Alternatively, a luminescent assay system or biosensor can be used to measure estrogen levels by incorporating human estrogen receptor α and/or β into a mammalian cell line in combination with an estrogen-responsive element (ERE) upstream of a luciferase gene reporter (Paris, et al., J. Clin. Endocrinol. Metab. 87: 791-797, 2002, which is incorporated herein by reference).

Levels of one or more estrogen receptors, estrogen receptor modulators, steroid hormones, or modulators or metabolites thereof can be measured using sensor technology, including for example, chemical sensors, biosensors, protein arrays, and/or microfluidic devices that can also be referred to as “lab-on-a-chip” systems (see, e.g., Cheng, et al., Anal. Chem. 73: 1472-1479, 2001; Bange, et al., Biosensors Bioelectronics 20: 2488-2503, 2005; De, et al., J. Steroid Biochem. Mol. Biol. 96: 235-244, 2005; Zhou, et al., Sci. China C. Life Sci. 49: 286-292, 2006; Hansen, et al., Nano Lett., 7: 2831-2834, 2007, which are incorporated herein by reference; Dauksaite et al., Nanotech 18(125503): 1-5, 2007). For example, a biosensor can be generated based on the interaction between estradiol and the estrogen receptor (see, e.g., Murata, et al., Anal. Sci. 17:387-390, 2001, which is incorporated herein by reference). In this instance, recombinant estrogen receptor is linked to an Au-electrode and cyclic voltammetric measurements are used to assess changes in the properties of the estrogen receptor protein layer in response to estradiol binding.

In some instances, the estrogen receptors, estrogen receptor modulators, steroid hormones, modulators, or metabolites thereof can be first extracted from the bodily fluid or tissue sample, e.g., bloody serum, plasma, urine, urogenital secretions, sweat and/or saliva, prior to performing one or more of the measurements described above. For example, a hormone, estradiol, can be extracted from serum using a combination of hexane and ethyl acetate followed by mixing, centrifugation, and collection of the organic layer (see, e.g., Dighe & Sluss, Clin. Chem. 50:764-6, 2004, which is incorporated herein by reference). Extracted hormones in the organic layer can be further fractionated using chromatography. For example, testosterone, dihydrotestosterone, androstenedione, estrone, and estradiol extracted from serum into an organic layer can be further fractioned using Celite column partition chromatography and eluting solvents such as toluene, isooctane and ethyl acetate (see, e.g., Hsing, et al., Cancer Epidemiol. Biomarkers Prev. 16:1004-1008, 2007, which is incorporated herein by reference). Radiolabeled internal standards corresponding to a given hormone can be used to assess procedural losses.

In some instances, steroid hormone levels or modulators or metabolites thereof in a subject can be measured transdermally using a non-invasive method, for example, reverse ionotophoresis. In general, iontophoresis is the application of a small electric current to enhance the transport of both charged and polar, neutral compounds across the skin. Reverse iontophoresis is the term used to describe the process whereby molecules are extracted from the body to the surface of the skin in the presence of an electrical current. The negative charge of the skin at buffered pH causes it to be permselective to cations causing solvent flow towards the anode. This flow is the dominant force allowing movement of neutral molecules across the skin. This technology can be used in devices for non-invasive and continuous monitoring of compounds in interstitial fluid of individuals with disease. See, e.g., Rhee, et al., J. Korean Med. Sci. 22:70-73, 2007; Sieg, et al., Clin. Chem. 50:1383-1390, 2004; which are incorporated herein by reference.

In some cases, data regarding physiological premenopausal levels of steroid hormone or modulators or metabolites thereof can be provided by an outside source. For example, a collection of physiological premenopausal levels can be provided as part of a patient's medical history. In addition to assessing the physiological premenopausal levels, the current physiologic levels of one or more steroid hormones can be assessed. These levels can be measured using any of the methods mentioned above, and can be a single measurement, or can include a collection of measurements taken, for example, over the course of one or more menstrual cycles or at more than one time of the day. In some cases, data regarding the current physiological levels can be provided by an outside source. For example one health care provider, such as a general practioner, can provide data to another user, e.g., a health care provider such as an endocrinologist.

The test results can be calculated or entered into a computer using a computer-readable medium. The computer would, for example, map out the monthly levels of steroid hormone and track changes in the monthly levels from one year to the next. As the levels of estrogens and progesterone decline from one testing period to the next, the computer program can calculate the difference between the current levels and baseline hormone levels and provide individualized dosage information for hormone replacement therapy to bring the current level of hormone up to the baseline physiologic level of hormone, maintaining the premenopausal level. For example, if the premenopausal level of estradiol of a female subject is approximately 75 pg/ml during the follicular phase of the menstrual cycle and falls to 50 pg/ml or below as the women enters perimenopause, sufficient estradiol or a metabolite can be administered to bring the blood level back to 75 pg/ml. The subject can also be prescribed progestin in doses specific to her needs. The dosing of each hormone is such that the total serum concentration of endogenous and exogenous hormone is equal to the baseline physiological concentration established during premenopause testing. In an example, a female subject can have a baseline serum level of estradiol measured at 70 pg/ml during the first week of her menstrual cycle, rising to over 200 pg/ml during ovulation and dropping back down to 70 pg/ml and for whom follow-up testing several years later determined the level of estradiol in the serum had fallen by 10%. In this case, sufficient exogenous estradiol can be administered to the female subject to replace the 10% and bring the serum levels back to the full baseline level. The amount of one or more estrogen receptor modulators, e.g., estrogen receptor α modulator, or estrogen receptor β modulator administered in combination with estradiol would depend upon the pharmacokinetics of the exogenous estradiol and the administration, for example, by oral, gel, transdermal or implanted route.

### Time-History of Serum Hormone Levels and Dosing

Prior to determining a treatment regimen, additional information regarding the physiological status of a mammalian subject can be gathered and assessed. For example, information on the mammalian subject's own history or his or her family's history of diseases, including genetic information, can be collected. The individualized medical evaluation can include a genetic profile of the mammalian subject regarding genes, genetic mutations, or genetic polymorphisms that can indicate risk factors that affect disease related to steroid hormone levels, hormone receptors, modulators, e.g., agonists or antagonists of estrogen receptors, steroid hormones or steroid hormone receptors, or factors causing genetic disease or a genetic predisposition to disease in the mammalian subject. The individualized treatment regimen includes at least one estrogen receptor modulator optionally in combination with replacement therapy for one or more steroid hormones, or metabolites or modulators thereof. The treatment regimen can be based upon information derived from pre-menopausal hormone levels or pre-disease hormone levels in the subject. In this context, a physiological level of a hormone includes the level of hormone measured at a given time. A physiological premenopausal level can be a level of the hormone as measured at a point in time during premenopause in a female subject. A physiological pre-disease level can be a level of the hormone as measured at a point in time prior to occurrence of disease or prior to surgery to treat a disease in a female or male subject. A current physiological level can be the level of the hormone as measured just prior to determining a treatment regimen. The levels of one or more estrogen receptors, estrogen receptor modulators, hormones, steroid hormones, modulators or metabolites thereof can be measured using the methods described herein to develop a time-history of serum hormone levels in a subject. A time-history of serum hormone levels of one or more estrogen receptors, estrogen receptor modulators, steroid hormones, or metabolites or modulators thereof in a subject refers to the level of one or more estrogen receptors, estrogen receptor modulators, steroid hormones, or metabolites or modulators thereof in the serum or tissue of a subject over time. As such, the level of one or more steroid hormones can be measured over any of a variety of time intervals. For example, a time-history of serum levels of one or more steroid hormone can be generated by measuring hormone levels over the course of one or more days, one or more weeks, one or more months, one or more years. In the case of a premenopausal or perimenopausal female subject, a time-history of serum levels of steroid hormones can be generated over the course of one or more menstrual cycle, for example, that can vary from 21 to 35 days. In the case of a male subject, a time-history of serum levels of steroid hormones, for example, testosterone, can be generated over the course of one or more years to assess seasonal variations in testosterone levels (see, e.g., Svartberg, et al., J. Clin. Endocrinol. Metab. 88: 3099-3104, 2003, which is incorporated herein by reference). In addition, a time-history of serum steroid hormone levels may not be contiguous in time. For example, steroid hormone levels can be measured 2-3 months out of a year, on a yearly basis, for example, and peak values or minimal values for the other months are inferred based on the average hormone levels during the measurement period. As such, a time-history of serum steroid hormone levels in a subject can be measured over multiple cycles and multiple monthly or yearly time periods.

The time-history of one or more estrogen receptors, estrogen receptor modulators, steroid hormones, metabolites or modulators thereof in serum or tissue of a mammalian subject can be stored, analyzed and tracked. Methods for storing this information include paper storage as well as electronic storage. Analysis and tracking can be done manually by looking at the data. A software program can be designed and used to store, analyze and track the time-history of serum steroid hormones of a subject. The software program can be used to monitor changes in the time-history of serum steroid hormone levels of a subject from one measurement period to the next. The software program can compare the time-history of serum steroid hormone levels of a subject relative to steroid hormone levels associated with an age-matched population norm. The software program can also compare the steroid hormone levels of a subject to a physiological level of hormone. The physiological level of one or more steroid hormone of a subject can be inferred by measuring hormone levels at a time in the subject's life when hormone levels are assumed to be within a “normal range.” For example, in the case of a female subject, this can be during premenopause. In the case of a male subject, this can be prior to the age of 40, for example. As such, time-history of serum steroid hormones can be used to monitor changes in levels of one or more steroid hormone relative to either a subject's own physiological level of hormone or that of a population norm. As hormone levels decline due to age, disease, or surgery, for example, supplemental hormone treatment can be used to treat cardiovascular disease or condition and to maintain the physiological level of one or more steroid hormones in the subject.

The physiological cyclic level of one or more estrogen receptors, estrogen receptor modulators, steroid hormones, metabolites or modulators thereof can be maintained by supplementing endogenous levels of steroid hormones with exogenous steroid hormones to bring the overall steroid hormone levels back to the physiological level. As such, the subject is dosed with sufficient supplemental steroid hormones, or metabolites, modulators, or analogs thereof to achieve the desired physiological level. It is anticipated that in the aging subject, the overall level of serum hormones can change over time, due, for example, to a decline in endogenous hormone even in the presence of exogenous hormone and/or to physiological changes in the subject such as a gain or loss of weight or onset of a systemic disease. As such, the hormone levels can be routinely measured, and a treatment regimen including replacement therapy for one or more estrogen receptors, estrogen receptor modulators, steroid hormones, or metabolites or modulators thereof, adjusted appropriately to treat cardiovascular disease or condition and to maintain the physiological steroid hormone level. The software program can be designed to include guidance regarding hormone dosing based on the measured differences in the overall hormone levels and the physiological levels.

Inferred peak values or minimal values of serum steroid hormone levels in the subject refers to steroid hormone levels in the subject that have been determined either by prior time-history of serum steroid hormone levels in the subject, a current time history, and/or by values of serum steroid hormone levels in a similar subject population determined by age, environment, family background, or genetic profile.

Treatment of Cardiovascular Disease or Condition with Treatment Regimen Including at Least One Estrogen Receptor Modulator Optionally in Combination with Replacement Therapy for One or More Steroid Hormones

A treatment regimen that includes at least one estrogen receptor modulator optionally in combination with replacement therapy for one or more steroid hormones, or metabolites or modulators thereof, to maintain a substantially physiological level in a mammalian subject can be used to treat a disease or symptoms associated with the loss of normal physiological hormone levels, e.g., a cardiovascular disease or condition. Such a loss in hormone levels can be associated with natural or surgically induced menopause or hypogonadism, for example. In women, menopause is defined as the last menstrual cycle and is characterized by a cessation of ovarian function, leading to a significant decline in the level of circulating estrogens. The period of declining ovarian function prior to menopause is termed perimenopause and can last for several years with fluctuating estrogen levels and erratic menstrual cycles. The changes in estrogen levels during perimenopause and at menopause can cause vasomotor symptoms such as hot flashes and palpitations, psychological symptoms such as depression, anxiety, irritability, mood swings and lack of concentration, atrophic symptoms such as vaginal dryness and urgency of urination, and skeletal symptoms such as osteopenia and muscle pain. Menopause can be induced by surgical removal of the ovaries. The symptoms associated with perimenopause, menopause, and post-menopause can be treated with estrogens either with or without a progestogen such as progestin. Progestin is added to the treatment regime, for example, to prevent estrogen-induced endometrial proliferation and cancer in women with intact uteri.

Menopause refers to the cessation of menstruation but it is commonly used to refer to the period in a woman's life when she passes out of her reproductive years. Menopause usually begins between the ages of 45 and 50, as the ovaries gradually cease to function and the production of the female sex hormones diminishes. The average age of menopause is 52 years of age. Perimenopause is a term used to describe the 5-15 years prior to the natural end of menstruation and is characterized by declining and fluctuating ovarian hormone production and as such is often associated with the physical symptoms of menopause such as hot flashes, increasing vaginal dryness, sleep problems, mood swings, and breast tenderness. Perimenopause typically begins between the ages of 35 and 50. The time period prior to perimenopause is referred to as premenopause.

Aging men also exhibit a natural decline in steroid hormones including, for example, decreased testosterone, estrone, androstanediol glucuronide, dehydroepiandrosterone, and dehydroepiandrosterone sulfate. For example, the normal levels of testosterone range from 270 to 1000 nanograms/deciliter in men under 40 but begin to decline on average 0.8%/year after the age of 40 (see, e.g., Feldman, et al., J. Clin. Endocrinol. Metab. 87:589-598, 2002, which is incorporated herein by reference). The decline in testosterone in aging men has been associated with parallel age declines in bone mass, muscle mass/strength, physical function/frailty, and sexual function with symptoms ranging from irritability, nervousness, anxiety, sweating, sleep disturbances, decreased energy, decreased beard growth, and decreased potency, morning erections, and libido. In addition, the reduction in testosterone can be linked with various age-associated metabolic changes such as abdominal obesity, diabetes, and markers of prediabetes (see, e.g, Araujo, et al., J. Clin. Endocrinol. Metab. 92:4241-4247, 2007, which is incorporated herein by reference). Testosterone levels can also decline or be absent all together (hypogonadism) for reasons other than aging. For example, hypogonadism in man can be due to problems with the testes themselves or with the pituitary gland. This can include disorders of the testes such as Klinefelter's syndrome, inflammation of the testes (orchitis), and dysfunction due to radiation or chemotherapy, and alcohol abuse. Removal of both testicles, injury to both testicles and undescended testicles are all causes of hypogonadism. Any disease of the pituitary gland can also result in hypogonadism. As such, supplemental testosterone therapy in the form of transdermal patches, gels and creams, for example, can be used to relieve the symptoms associated with the decline or lack of testosterone (see, e.g., Bain Canadian Family Physician 47:91-97, 2001, which is incorporated herein by reference).

In addition to relieving the symptoms associated with age-, disease- or surgery-related decrease in one or more steroid hormones, maintaining a substantially physiological level of one or more steroid hormones, can be of use in preventing or slowing the onset or progression of disease, for example, cardiovascular disease or condition.

A treatment regimen including at least one estrogen receptor modulator optionally in combination with one or more steroid hormones, metabolites or modulators thereof, to maintain substantially physiological cyclic levels of one or more steroid hormones, or metabolites or modulators thereof, can be used to treat cardiovascular disease or condition in a mammalian subject. For example, a treatment regimen determined based on the APOE allelic profile in a subject and based on a subject's physiological pre-menopausal hormone levels and current physiological hormone levels can include providing an amount and type of estrogen receptor modulator and/or an amount and type of estrogen, administered in a particular fashion, to a woman whose estrogen levels only recently decreased, such as a woman who is transitioning into menopause or in early menopause, or a woman has recently lost ovary function due to surgery, exposure, or disease. In animals and humans endogenous and exogenously provided estrogen is protective against atherogenesis and cardiovascular disease or condition in general, especially in younger women. For example, later age of menopause and longer exposure to endogenous estrogens is associated with protection against cardiovascular disease or condition (see van der Schouw et al., Lancet 16:714-8 1996, which is incorporated herein by reference), and premature atherosclerosis common in women and primates with premenopausal estrogen deficiency can be prevented by estrogen treatment (see Clarkson, Menopause 14: 373-84, 2007, which is incorporated herein by reference). Studies in animals also demonstrated that the timing of initiation of estrogen treatment (e.g., 17β-estradiol, E2) after loss of ovarian hormone function in a subject is a major indicator for successful therapeutic cardiovascular outcomes (Pinna et al., Hypertension 51: 1210-1217, 2008). In the estrogen-only arm of the WHI trial, an analysis of the 50-59-year-old age group showed a near statistical decrease in coronary events: 63 (0.36-1.08), and a statistically significant reduction in a global coronary score, 0.66 (0.45-0.96) (see Hsia, et al., Arch Intern Med. 2006; 166:357-365). Current therapy and Clinical Trials (see, e.g., The Kronos Early Estrogen Prevention Study (KEEPS)) are now focusing on treating women transitioning into menopause and early menopause with estrogens alone or in combination administered orally or transdermally (Harman, et al., Climacteric 8(1):3-12, 2005; Miller et al., J. Appl. Physiol. 99: 381-383, 2005; which are incorporated herein by reference.). Women, for example young women with estrogen deficiency due to oophorectomy, disease or early menopause, as well as women transitioning through menopause can benefit in protection against cardiovascular disease or condition by a treatment method for cardiovascular disease or condition that includes providing a treatment regimen including at least one estrogen receptor modulator optionally in combination with replacement therapy for one or more steroid hormones or metabolites or modulators thereof. The treatment regimen includes selecting a single specific form of estrogen receptor modulator, e.g., estrogen receptor α modulator, or estrogen receptor β modulator, a single specific form of estrogen, e.g., a natural estradiol, and/or a particular means of delivery, e.g., transdermal delivery, and/or including providing an antagonistic tissue-specific estrogen receptor modulator (SERM) to inhibit responses in certain tissues. (Qiao, et al. in Gender Medicine. 5: Suppl. A, S46-S64, 2008, which is incorporated herein by reference.) In another example, progesterone therapy, for example as part of a treatment regimen including replacement therapy for one or more steroid hormones or modulators or metabolites thereof, can be useful for treatment of inflammatory disorders and as a cardioprotective agent against reperfusion injury resulting from a myocardial ischemia. See, e.g. Booth et al., Am J Physiol Heart Circ Physiol, 293: H1408-H1415, 2007; Booth et al., J Pharmacol Exp Ther, 307: 395-401, 2003, which are incorporated herein by reference.

### Genetic Profiling

Prior to determining a treatment regimen, additional information regarding the physiological status of a mammalian subject can be gathered and assessed. For example, information on the subject's own history or his or her family's history of diseases, including genetic information, can be collected. The medical evaluation can include a genetic profile of the subject regarding genes, genetic mutations, or genetic polymorphisms that can indicate risk factors that affect disease and/or are related to steroid hormone levels, hormone receptors, modulators, e.g., agonists or antagonists, of steroid hormones or steroid hormone receptors, or factors causing genetic disease or a genetic predisposition to disease in the subject. Medical evaluation regarding genetic profiling or genetic testing can be provided as a current determination of genetic risk factors, or as part of the subject's medical history. Genetic profiling or genetic testing can be used to design a treatment regimen and determine an optimal level individualized for the subject of at least one estrogen receptor modulator optionally in combination with one or more steroid hormones, steroid hormone receptors, metabolites, or modulators thereof, wherein the genetic profile was obtained during a pre-menopausal or pre-disease period from the subject. A physician can use the genetic profiling or genetic testing information to determine a genetic basis to treat cardiovascular disease or condition and to maintain a substantially physiological cyclic level of one or more steroid hormones in a mammalian subject in need thereof. Determining a genetic profile of a subject can be used to predict the potential response to a treatment regimen designed to treat cardiovascular disease or condition and to maintain physiological cyclic levels of one or more steroid hormones, or modulators or metabolites thereof. In addition, genetic profiling of a subject can predict the risk of developing a chronic or life threatening disease that can be attenuated or prevented by providing a treatment regimen including at least one estrogen receptor modulator optionally in combination with one or more steroid hormones, or metabolites or modulators, or analogs thereof. In general, genetic profiling refers to analysis of a subject's genomic DNA for the purpose of comparing with known genetic information.

Information gathering can include screening the subject for the presence of disease and/or undertaking genetic profile screening. For example, the subject can be screened for specific diseases or conditions and information used in the determining of the treatment regimen. For individuals with a known history or who are at risk of cardiovascular disease, a treatment regimen can be determined using one or more estrogen receptor modulators, e.g., an estrogen receptor α modulator or an estrogen receptor β modulator, in combination with a progestogen in addition to one or more estrogens in order to treat cardiovascular disease and to maintain the physiological levels of the steroid hormones. In a further embodiment, for a female subject with multiple risk factors for heart disease such as diabetes, high blood pressure, a strong family history or genetic predisposition to disease, a treatment regimen for cardiovascular disease can be determined that includes selecting one or more estrogen receptor modulators, e.g., estrogen receptor α modulator, or estrogen receptor β modulator optionally in combination with a single specific form of estrogen or metabolite, modulator, mimetic, or analog thereof, and/or a particular means of delivery, such as transdermal. Selectivity of one or more estrogen receptor modulators, e.g., estrogen receptor α modulators or estrogen receptor β modulators, have been discussed. Selectivity of steroid hormones and receptors and its manipulation are discussed by Qiao, et al. in Gender Medicine. 5: Suppl. A, S46-S64, 2008, which is incorporated herein by reference.

A genetic polymorphism or genetic mutation in a genetic profile of a mammalian subject that encodes a component of one or more steroid hormone signaling pathway can affect the levels of the levels of hormones and related compounds. As such, genetic profiling can be used prior to the initiation of a treatment regimen including at least one estrogen receptor modulator optionally in combination with one or more steroid hormones, or metabolites, modulators, or analogs thereof, to assess whether the subject has any genetic mutations and/or genetic polymorphisms that can be historically correlated with levels of at least one estrogen receptor modulator optionally in combination with one or more steroid hormones, or metabolites, modulators, or analogs thereof. In humans, genetic profiling of the three alleles of APOE (ε2, ε3, and ε4) including 6 different genotypes (ε2/ε2, ε2/ε3, ε2/ε4, ε3/ε3, ε3/ε, and ε4/ε4) can be used to associate an APOE allelic profile with the incidence of cardiovascular disease or condition in a subject. For example, a method for treating cardiovascular disease or condition in a mammalian subject can include at least one treatment regimen to the subject including at least one estrogen receptor modulator, wherein the at least one treatment regimen is determined based on the APOE allelic profile in the subject. In an aspect, the subject's APOE profile is at least one of APOE ε2 positive and APOE ε3 positive, and the at least one estrogen receptor modulator includes at least one selective estrogen receptor α agonist. In a further aspect, the subject's APOE profile is ε4 positive, and the at least one treatment regimen including at least one selective estrogen receptor β agonist.

A polymorphism or mutation in the genetic information of a mammalian subject that encodes a component of one or more steroid hormone signaling pathway can dictate how well that subject will respond to treatment with one or more steroid hormones, or metabolites, modulators, or analogs thereof. Genetic profiling can be used prior to the initiation of a treatment regimen including one or more steroid hormones, or metabolites, modulators, or analogs thereof to assess whether the subject has any genetic mutations and/or polymorphisms that can be historically correlated with a positive or negative response to a treatment regimen to treat cardiovascular disease or condition and to maintain physiological cyclic levels of one or more steroid hormones, or metabolites, or modulators thereof in the subject. Of particular interest are potential mutations or polymorphisms associated with either hormone receptors or other components of the hormone signaling pathway that can differentially respond to supplemental hormone treatment. Receptors that would be of interest for genetic profiling can include but are not limited to the estrogen receptors (ERα and ERβ), the androgen receptor (also known as NR3C4, nuclear receptor subfamily 3, group C, member 4), the progesterone receptor (also known as NR3C3, nuclear receptor subfamily 3, group C, member 4), the follicle stimulating hormone receptor (FSHr), luteinizing hormone receptor (LHr), and anti-Mullerian receptor type II. The complete genomic DNA sequence as well as the coding DNA sequence for these and other relevant targets can be found, for example, in the National Center for Biotechnology Information (NCBI) database (http://www.ncbi.nlm.nih.gov/).

A number of genetic polymorphisms have been studied in association with the estrogen receptor. For example, a single nucleotide polymorphism in the estrogen receptor α gene confers positive changes in lumbar bone mineral density following hormone replacement therapy (see, e.g., Yahata, et al., Hum. Reprod. 20:1860-1866, 2005, which is incorporated herein by reference). In this instance, women with the non-coding genotype IVS6+141441 showed significant increases in bone mineral density ranging on average from 5.0 to 8.0% in each year of three years of hormone replacement therapy relative to women lacking the IVS6+141441 genotype. Polymorphisms in the estrogen receptor can also confer positive changes in HDL cholesterol levels in response to hormone replacement therapy (see, e.g., U.S. Pat. No. 6,828,103, which is incorporated herein by reference).

Medical evaluation of the mammalian subject for genetic profiling or genetic testing to determine genetic polymorphisms can be provided as a current determination of genetic risk factors in the mammalian subject, or as part of the subject's medical history. In some instances, polymorphisms in the estrogen receptor or other hormone receptor can be associated with an increased risk of developing a specific disease, that can inform a physician as to whether or not hormone treatment would be appropriate for a particular subject. For example, polymorphisms in estrogen receptor β gene variants are associated with increased risk of Alzheimer's disease in women (see, e.g., Priskanen, et al., Eur. J. Hum. Genet. 13:1000-1006, 2005, which is incorporated herein by reference). Similarly, the severity of cardiovascular disease or condition in both men and women can be correlated with polymorphisms in the estrogen receptor α gene. For example, severity of coronary artery disease in postmenopausal women, as judged by the number of vessels with 50% stenosis, was greater in women carrying the PvuII CT genotype and the XbaI GA genotype relative to the other PvuII and XbaI genotypes (see, e.g., Alevizaki, et al., Eur. J. Endocrinol. 156:489-496, 2007, which is incorporated herein by reference).

Genetic polymorphisms in a hormone receptor gene can predict relative response to a given hormone in terms of how well the receptor uses available hormone and how well the resulting signaling events are transmitted. For example, the PROGINS polymorphisms in the human progesterone receptor reduce the stability of the receptor mRNA transcript, reduce transactivation activity of the receptor, and reduce the efficiency of progestin-induced inhibition of cell proliferation (see, e.g., Romano, et al., J. Mol. Endocrinol. 38:331-350, 2007, which is incorporated herein by reference). Women who carry the PROGRINS polymorphisms are at increased risk for developing ovarian cancer, endometrial cancer and endometriosis. In another example, polymorphisms in the follicle stimulating hormone receptor resulting in a single amino acid change from asparagine to serine results in lower sensitivity to FSH, decreased negative feedback and longer menstrual cycles (see, e.g., Greb, et al., J. Clin. Endocrinol. Metab. 90:4866-4872, 2005, which is incorporated herein by reference).

Genetic profiling of potential disease markers or risk indicators can be used as part of treating a mammalian subject with at least one estrogen receptor modulator optionally in combination with one or more steroid hormones, or metabolites, modulators, or related compounds to treat cardiovascular disease or condition and to maintain a physiological level of the steroid hormones. Genetic profiling can be used to assess whether or not a subject is at risk for developing a chronic or life threatening disease that can be attenuated or prevented by use of supplemental hormone treatment. Chronic or life threatening diseases of interest can include but are not limited to cardiovascular disease or condition, heart disease, bleeding, inflammation, atherosclerosis, hypercholesterolemia, acute myocardial infarction, angina pectoris, myocardial ischemia, reperfusion injury, venous thrombosis, coronary insufficiency, coronary valve disease, coronary valve stenosis, coronary heart disease, valve disease, atherothrombotic stroke, or intermittent claudication. Valve disease or disorder can include, but is not limited to, mitral valve insufficiency, mitral valve prolapse, mitral valve stenosis, tricuspid valve insufficiency, and tricuspid valve stenosis. See, e.g., Colao, et al., Eur. J. Endocrinology 151: S93-S101, 2004; JAMA 301: 1892-1901, 2009, which are incorporated herein by reference. For example, development of neurological disease, e.g., late-onset Alzheimer's disease is associated with a specific polymorphism in apolipoprotein E (APOE) termed the ε4 genotype. See, e.g., Strittmatter, et al., Proc. Natl. Acad. Sci., USA., 90:1977-1981, 1993, which is incorporated herein by reference. As another example, a polymorphism in the NEDD9 gene (neural precursor cell expressed, developmentally down-regulated gene) has been correlated with an increased risk of developing late-onset Alzheimer's disease and Parkinson's disease with odd ratios of 1.38 (1.20-1.59) and 1.31 (1.05-1.62), respectively. See, e.g., Yonghong, et al., Hum. Mol. Genet. 17: 759-767, 2008, which is incorporated herein by reference.

Genetic profiling can be used to identify mammalian subjects with a predisposition to hypertension or other cardiovascular disease or condition. For subjects determined to have a predisposition to hypertension and/or other cardiovascular disease or condition, a treatment regimen including replacement therapy for one or more steroid hormones or metabolites or modulators thereof, can be developed to reduce the incidence of hypertension and/or cardiovascular disease or condition, for example in a female subject. These diseases can develop during transitions from premenopause to perimenopause, early menopause, late menopause, and/or post menopause. Qiao et al., Gender Medicine 5: Suppl. A, S46-S64, 2008, which is incorporated herein by reference.

Polymorphisms or mutations in a number of genes have been linked to increased risk of developing osteoporosis in mammalian subjects. These can include but are not limited to genes encoding lipoprotein receptor-related protein 5 (LRP5), transforming growth factor β 1 (TGF-β1), bone morphogenic proteins (BMPs), sclerostin, CBFA1 gene, cathespin K, TCIRG1 gene, CLCN7 gene, Vitamin D receptor, collagen types Iα I (COLIA1), and estrogen receptor α (see, e.g., Ralston and decrombrugghe, Genes & Dev. 20:2492-2506, 2006, which is incorporated herein by reference). For example, amino acid substitutions Ala1330Val and Val667Met in LRP5 increase the risk of developing osteoporosis in older men with odds ratios ranging from 1.01 to 8.81 (see, e.g., Brixen et al, Calcif. Tissue Int. 81:421-429, 2007, which is incorporated herein by reference).

Genomic DNA for use in genetic profiling can be isolated from any biological sample that contains the DNA of that mammalian subject, including but not limited to saliva, cheek swab, blood, or other tissue. For example, genomic DNA can be extracted from whole blood or from isolated peripheral blood leukocytes isolated by differential centrifugation from whole blood using a commercially available DNA purification kit (see, e.g., QIAamp DNA Blood Mini kit, Qiagen, Valencia, Calif.) using the manufacturer's instructions.

Medical evaluation of the mammalian subject for genetic profiling or genetic testing can be provided as a current determination of genetic risk factors in the subject, or as part of the subject's medical history. Genetic profiling or genetic testing can be carried out using a variety of methods including but not limited to restriction landmark genomic scanning (RLGS), southern blot analysis combined with restriction fragment length polymorphism (RFLP), fluorescence in situ hybridization (FISH), enzyme mismatch cleavage (EMC) of nucleic acid heteroduplexes, ligase chain reaction (LCR), and polymerase chain reaction (PCR) based methods (Tawata, et al., Comb. Chem. High Throughput Screen. 3:1-9, 2000, which is incorporated herein by reference). Analysis of one or more single nucleotide polymorphisms (SNPs) can also be used for genetic profiling.

Restriction fragment landmark genomic scanning (RLGS) can be used to scan an entire mammalian genome. As such, genomic DNA is digested with restriction enzymes to generate large DNA fragments. The fragments are separated on an agarose gel, digested with one or more restriction enzymes within the agarose gel, and then separated in a second dimension by polyacrylamide gel electrophoresis (PAGE) (Tawata, et al., Comb. Chem. High Throughput Screen. 3:1-9, 2000, which is incorporated herein by reference). The DNA can be labeled prior to digestion, or the fragments can be stained nonspecifically as with an intercalating dye, for example. The resulting pattern can be compared with pre-established norms to detect genetic mutations.

Restriction fragment length polymorphism (RFLP) is similar to restriction fragment landmark genomic scanning in that the genomic DNA is digested with specific restriction enzymes and separated on an agarose gel. The separated DNA is transferred to a membrane and the fragments are visualized using hybridization analysis and gene specific probes.

A variety of PCR related methods can be used for genetic profiling and can be used to detect both known and unknown mutations and polymorphisms (Tawata, et al., Comb. Chem. High Throughput Screen. 3:1-9, 2000, which is incorporated herein by reference). For known mutations and polymorphisms, specific PCR oligonucleotide probes are designed to bind directly to the mutation or polymorphism or proximal to the mutation or polymorphism. For example, PCR can be used in combination with RFLP. In this instance, a DNA fragment or fragments generated by PCR with primers on either side of the mutation or polymorphism site are treated with restriction enzymes and separated by agarose gel electrophoresis. The fragments themselves can be detected using an intercalating dye, for example, ethidium bromide. An aberrant banding pattern can be observed if mutations exist within the restriction sites. PAGE can be used to detect single base differences in the size of a fragment.

Alternatively, PCR can be used in combination with DNA sequencing for genetic profiling. For example, PCR primers can be designed that bind to either side of a potential mutation site on the target DNA and generate a PCR fragment that spans a potential mutation site. The PCR fragment is either directly sequenced or subcloned into a cloning vector and subsequently sequenced using standard molecular biology techniques.

Alternatively, a mutation or polymorphism can be screened using comparative genomic hybridization (CGH) (Pinkel & Albertson, Nature Gen. 37:S11-S17, 2005, which is incorporated herein by reference). In this instance, “normal” genomic DNA and test genomic DNA are differentially labeled and hybridized to metaphase chromosomes or DNA microarrays. The relative hybridization signal at a given location is proportional to the relative copy number of the sequences in the reference and test genomes. Arrays can be generated using DNA obtained from, for example, bacterial artificial chromosomes (BACs) or PCR.

Analysis of one or more single nucleotide polymorphism (SNP) can be used for genetic profiling. A SNP is a DNA sequence variation in which a single nucleotide in the genomic sequence differs between members of a species (or between paired chromosomes of an individual). For a variation to be considered a SNP it must occur in at least 1% of the population. Most SNPs do not affect protein function, and/or are not responsible for a disease state, but they can serve as biological markers for pinpointing a region associated with an altered protein or a disease on the human genome map, as SNPs are often located near a gene found to be associated with a certain disease. Occasionally, a SNP can actually affect protein function and/or cause a disease and, therefore, can be used to search for and isolate a specific gene, e.g., a T to C mutation in the CYP17 gene that affects enzyme function. The pattern of SNPs in a subject's genomic DNA can be compared with information in databases in an association study to determine effect on protein function and/or risk of disease development. SNPs can be identified using PCR and DNA sequencing as described above. Alternatively, SNP genotyping can be done using high throughput array analysis (see, e.g., Applied BioSystems, ABI PRISM, 3100 Genetic Analyzer with 22-cm Capillary Array; Syvanen, et al., Nature Genetics, 37: S5-S10, 2005, which is incorporated herein by reference). A growing number of web-based databases are available for finding information regarding SNPs and protein function and/or disease associations (see, e.g., International HapMap Project: http://snp.cshl.org/; Nature 449: 851-861, 2007; National Center Biotechnology Information (NCBI) Single Nucleotide Polymorphisms, http://www.ncbi.nlm.nih.gov/projects/SNP/.)

### Drug Delivery/Time Release Device

A treatment regimen that includes at least one estrogen receptor modulator optionally in combination with one or more steroid hormones, or metabolites, modulators, or analogs thereof for treatment of cardiovascular disease or condition in a mammalian subject can be administered to the mammalian subject by a variety of methods, for example, via oral, parenteral, subcutaneous, intravenous, intramuscular, intraperitoneal, transdermal, transbuccal, intraocular, or intravaginal routes, e.g., by inhalation, intra-nasal spray, by depot injections, or by hormone implants.

Pharmaceutical compositions containing at least one estrogen receptor modulator optionally in combination with one or more steroid hormones or metabolites, modulators, or analogs thereof and a suitable carrier can be solid dosage forms that include, but are not limited to, tablets, capsules, cachets, pellets, pills, powders and granules; topical dosage forms that include, but are not limited to, solutions, powders, fluid emulsions, fluid suspensions, semi-solids, ointments, pastes, creams, gels and jellies, foams and patches; and parenteral dosage forms that include, but are not limited to, solutions, suspensions, emulsions, and dry powders.

The administration of at least one estrogen receptor modulator optionally in combination with one or more steroid hormones, or metabolites, modulators, or analogs thereof to a mammalian subject can constitute a single dose, multiple daily doses, multiple doses per day, continuous infusion and or time released dose. A cyclic, continuous or combination dosing regime can be used. For example, estrogen can be taken for 25 days each month with progestin added for 10 to 12 days, and no medication used for 3 to 6 days per month. Menstrual-like bleeding is expected during the period when no medication is taken in women who have not reached menopause. Alternatively, estrogen can be given daily with progestin added for 10 to 14 days per month. Alternatively, estrogen and progestin can be given continuously as a daily oral tablet.

At least one estrogen receptor modulator optionally in combination with one or more steroid hormones, or metabolites, modulators, or analogs thereof can be administered orally using, for example, push-fit capsules made of gelatin or soft sealed capsules made of gelatin and a plasticizer such as glycerol or sorbitol. One or more steroid hormone can be combined with fillers such as, e.g., lactose, binders such as, e.g., starches, and/or lubricants such as, e.g., talc or magnesium stearate and, optionally, stabilizers. In soft capsules, one or more steroid hormone can be dissolved or suspended in suitable liquids, such as fatty oils, liquid paraffin, or liquid polyethylene glycols. In addition, stabilizers can be added.

At least one estrogen receptor modulator optionally in combination with one or more steroid hormones, or metabolites, modulators, or analogs thereof can be administered to the mammalian subject by inhalation using an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount.

At least one estrogen receptor modulator optionally in combination with one or more steroid hormones, or metabolites, modulators, or analogs thereof can be formulated for parenteral administration to the mammalian subject by injection, e.g., by bolus injection or continuous infusion. At least one estrogen receptor modulator optionally in combination with one or more steroid hormones can be administered by continuous infusion subcutaneously over a period of about 15 minutes to about 24 hours. In some instances, continuous infusion can be done over the course of days and/or months. Compositions for injection can be presented in unit dosage form, e.g., in ampoules or in multi-dose containers, with an added preservative. The compositions can take such forms as suspensions, solutions or emulsions in oily or aqueous vehicles, and can contain agents such as suspending, stabilizing and/or dispersing agents.

### Transdermal Delivery Method

In general, at least one estrogen receptor modulator optionally in combination with at least one treatment regimen including at least one replacement therapy including one or more steroid hormones or metabolites or modulators thereof for treatment of cardiovascular disease or condition in the mammalian subject can be delivered through or across the skin of the subject using either passive or active transdermal delivery methods. Passive transdermal delivery methods utilize passive diffusion of agents across the skin and are exemplified by adhesive transdermal patches. In this instance, a patch is applied to the skin of the subject and one or more steroid hormones slowly and continuously diffuses out of the patch at a rate dictated by the formulation of one or more steroid hormone and the composition of the patch. For example, transdermal administration of estrogen is known in the art and described in U.S. Pat. Nos. 4,460,372; 4,573,996; 4,624,665; 4,722,941; and 5,223,261; which are incorporated herein by reference.

A transdermal patch for administering at least one estrogen receptor modulator optionally in combination with one or more steroid hormones, or metabolites, modulators, or analogs thereof can include a non-permeable backing layer, a permeable surface layer, an adhesive layer, and a reservoir containing hormone as described in U.S. Patent Publication 2008/0119449, which is incorporated herein by reference.

Examples of suitable materials that can comprise the non-permeable backing layer are well known in the art of transdermal patch delivery and can include, but are not limited to, polyester film, such as high density polyethylene, low density polyethylene or composites of polyethylene; polypropylene; polyvinyl chloride, polyvinylidene chloride; ethylene-vinyl acetate copolymers; and the like.

Examples of suitable permeable surface layer materials are also well known in the art of transdermal patch delivery, and any conventional material that is permeable to the one or more steroid hormone to be administered, can be employed. Specific examples of suitable materials for the permeable surface layer can include, but are not limited to, dense or microporous polymer films such as those comprised of polycarbonates, polyvinyl chlorides, polyamides, modacrylic copolymers, polysulfones, halogenated polymers, polychloroethers, acetal polymers, acrylic resins, and the like (see, e.g., U.S. Patent Publication 2008/0119449, which is incorporated herein by reference).

Examples of suitable adhesives that can be coated on the backing layer to provide the adhesive layer are also well known in the art and can include, for example pressure sensitive adhesives such as those comprising acrylic and/or methacrylic polymers. Specific examples of suitable adhesives can include polymers of esters of acrylic or methacrylic acid (e.g., n-butanol, n-pentanol, isopentanol, 2-methyl butanol, 1-methyl butanol, 1-methyl pentanol, 3-methyl pentanol, 3-methyl pentanol, 3-ethyl butanol, isooctanol, n-decanol, or n-dodecanol esters thereof) alone or copolymerized with ethylenically unsaturated monomers such as acrylic acid, methacrylic acid, acrylamide, methacrylamide, N-alkoxymethyl acrylamides, N-alkoxymethyl methacrylamides, N-t-butylacrylamide, itaconic acid, vinyl acetate, N-branched C.sub.10-24 alkyl maleamic acids, glycol diacrylate, or mixtures of the foregoing; natural or synthetic rubbers such as silicon rubber, styrene-butadiene rubber, butyl-ether rubber, neoprene rubber, nitrile rubber, polyisobutylene, polybutadiene, and polyisoprene; polyurethane elastomers; vinyl polymers such as polyvinyl alcohol, polyvinyl ethers, polyvinyl pyrrolidone, and polyvinyl acetate; ureaformaldehyde resins; phenol formaldehyde resins; resorcinol formaldehyde resins; cellulose derivatives such as ethyl cellulose, methyl cellulose, nitrocellulose, cellulose acetatebutyrate, and carboxymethyl cellulose, and natural gums such as guar, acacia, pectin, starch, destria, gelatin, casein, and the like.

A treatment regimen including at least one estrogen receptor modulator optionally in combination with one or more steroid hormones, or metabolites, modulators, or analogs thereof can be administered by active transdermal delivery methods that utilize an energy source to increase the flux of the one or more steroid hormone across the skin either by altering the barrier function of the skin (primarily the stratum corneum) or by increasing the energy of the hormone molecules. In this instance, the level of at least one estrogen receptor modulator optionally in combination with one or more steroid hormones delivered through the skin to the subject can be proportional to the overall level of energy applied.

Energy sources for use in active transdermal delivery can include, but are not limited to, electrical (e.g., iontophoresis and electroporation), ultrasonic (phonophoresis, sonophoresis), magnetic (magnetophoresis), and thermal energies (see, e.g., Gordon, et al., “Transdermal Delivery: 4 Myths about transdermal drug deliver”, Drug Delivery Technology, 3(4): June 2003 which is incorporated herein by reference). Iontophoresis, for example, uses low voltage electrical current to drive ionized agents or drugs across the skin. An electric current flows from an anode to a cathode, with the skin completing the circuit and drives ionized molecules into the skin from a reservoir associated with the transdermal delivery device. By contrast, electroporation uses short electrical pulses of high voltage to create transient aqueous pores in the skin through which an agent or drug can be transported. Phonophoresis or sonophoresis uses low frequency ultrasonic energy to disrupt the stratum corneum. For example, studies have described enhanced systemic levels of topical dexamethasone when applied in combination with ultrasound pulsed with an intensity of 1.0 W/cm² at a frequency of 3-MHz for 5 minutes (Saliba, et al., J. Athletic Training. 43:349-354, 2007, which is incorporated herein by reference). Thermal energy can be used to facilitate transdermal delivery by making the skin more permeable and by increasing the energy of drug molecules. In addition, one or more chemical permeation enhancer can be included. Examples of such enhancers can include, but are not limited, to isopropyl myristate, bile salts, surfactants, fatty acids and derivatives, chelators, cyclodextrins or chitosan.

In some aspects, transdermal delivery of at least one estrogen receptor modulator optionally in combination with one or more steroid hormones or metabolites, modulators, or analogs thereof can be facilitated using microporation induced by an array of microneedles. Microneedles, when applied to the skin, painlessly create micropores in the stratum corneum without causing bleeding and lower the resistance to drug diffusion through the skin. The microneedles can be used to abrade or ablate the skin prior to transdermal transport of one or more steroid hormone. For example, a micro-array of heated hollow posts can be used to thermally ablate human skin in preparation for transdermal drug delivery by diffusion as described in U.S. Patent Application 2008/0045879, which is incorporated herein by reference. Alternatively, an array of microfine lances or microneedles can be designed to actively inject drug into the skin as described in Roxhed, et al., IEEE Transactions on Biomedical Engineering, 55:1063-1071, 2008, which is incorporated herein by reference.

In some aspects, transdermal delivery of at least one estrogen receptor modulator optionally in combination with one or more steroid hormones, or metabolites, modulators, or analogs thereof facilitated by an energy source can be combined with a method that perforates or abrades the skin of a subject. For example, a transdermal delivery method can combine iontophoresis with one or more microprojections that perforate the skin and enhance penetration and delivery of an agent as described, for example, in U.S. Pat. No. 6,835,184 and U.S. Patent Application 2006/0036209, which are incorporated herein by reference. In another example, an energy source such as iontophoresis or electroporation can be combined with electrically-induced ablation of skin cells as described in U.S. Pat. No. 7,113,821, which is incorporated herein by reference.

A treatment regimen including at least one estrogen receptor modulator optionally in combination with one or more steroid hormones, or metabolites, modulators, or analogs thereof can be delivered to a subject by a transdermal delivery method by one or more functional modes, for example, completely automatic with a preset dosage regimen, controlled by the subject or other individual, or automatically controlled by a feedback mechanism based the normal physiological level of the hormones. For example, a preset dosage regimen of exogenous hormones can be administered to a subject to supplement endogenous hormone levels to bring the latter to physiologically normal levels. As such, a transdermal delivery system can be designed that automatically times the activation and deactivation of an electrical power supply, for example, for delivery and cessation of delivery of a drug at a variable controlled rate at preset or preprogrammed time intervals as described in U.S. Pat. No. 5,224,928, which is incorporated herein by reference. The pre-set dosage regimen can be programmed into the transdermal delivery method at the time of manufacture. Alternatively, the transdermal delivery method can have a removable computer interface component that can be externally programmed for a specific drug delivery regimen and reinserted into the device such as described in U.S. Pat. No. 6,539,250, which is incorporated herein by reference.

In a further aspect, a treatment regimen including at least one estrogen receptor modulator optionally in combination with one or more steroid hormones, or metabolites, modulators, or analogs thereof can be delivered to a subject by a transdermal delivery method, parenteral delivery method, or oral or nasal delivery method by one or more functional modes, for example, automatically controlled by a feedback mechanism based on the normal physiological level of the hormones. Close control of steroid hormone levels significantly reduces complications in treatment or prevention of hormone-related diseases. A control method for the automation of steroid hormone infusion that utilizes emerging technologies in blood or tissue steroid hormone biosensors is presented. The controllers that have been developed provide tighter, more optimal control of blood or tissue steroid hormone levels, while accounting for variation in patient response, estrogen receptor modulator or steroid hormone employed, or metabolite, modulator, or derivative thereof, and sensor bandwidth. Particular emphasis can be placed on controller simplicity and robustness necessary for medical devices and implants. In an example controlling blood glucose levels to treat a subject with type I diabetes, a PD controller with heavy emphasis on the derivative term is found to outperform the typically used proportional-weighted controllers in glucose tolerance and multi-meal tests. Dudde, et al., IEEE Trans Inf Technol Biomed. 10: 395-402, 2006, which is incorporated herein by reference. Suitability can be investigated of existing wearable continuous steroid hormone infusors controlled and adjusted by a control algorithm using continuous steroid hormone measurements as input to perform the functionality to maintain the normal physiological level of the hormones. Special attention can be given to the development of a continuous steroid hormone monitor and to evaluate which quality of input data is necessary for the control algorithm. Lam et al., Med Eng Phys. 24: 663-672, 2002, which is incorporated herein by reference.

In some instances, the transdermal delivery of a treatment regimen including at least one estrogen receptor modulator optionally in combination with one or more steroid hormones, or metabolites, modulators, or analogs thereof can be controlled either by the subject or other individual, for example, a healthcare provider, using on/off and/or high/low settings, for example, as described in U.S. Pat. No. 5,224,927, which is incorporated herein by reference. In some instances, it can be of benefit to limit or regulate the number of doses allowed by the subject. As such, the transdermal delivery method can incorporate a preprogrammed number of doses allowed during a given time period.

### Implantable Delivery Method

In general, a treatment regimen for cardiovascular disease or condition can be delivered systemically and/or to a specific site of action using an implantable delivery method. An implantable delivery method can incorporate a polymer or other matrix that allows for passive and slow release of a treatment regimen including at least one estrogen receptor modulator optionally in combination with one or more steroid hormones, or metabolites, modulators, or analogs thereof as exemplified, for example, by subcutaneous contraceptive implants. For example, a biologically active compound can be formulated with a solid hydrophilic polymer that swells by osmotic pressure after implantation, allowing interaction with a solubilizing agent and release of the biologically active compound through a non-porous rate-controlling membrane as described in U.S. Pat. No. 5,035,891, which is incorporated herein by reference. Alternatively, at least one estrogen receptor modulator optionally in combination with one or more steroid hormones, or metabolites, modulators, or analogs thereof can be delivered using an implantable delivery method that includes an infusion pump that actively moves the one or more steroid hormones from an associated reservoir into a subject. A variety of pumps can be incorporated into an implantable delivery method, for example, a piston pump, rotary vane pump, osmotic pump, Micro Electro Mechanical Systems (MEMS) pump, diaphragm pump, peristaltic pump, or solenoid piston pump. For example, the infusion pump can be a vapor-pressure powered pump in which a fluorocarbon charging fluid such as freon is used to drive the pump as a vapor-liquid mixture at normal body temperature and atmospheric pressure. Alternatively, the infusion pump can be a battery operated peristaltic pump. The latter is exemplified by an intrathecal drug delivery device in which an infusion pump with a controllable receiver unit is implanted under skin and a catheter is fed into the target site, in this case the spine (see, e.g., Belverud, Neurotherapeutics. 5:114-122, 2008, which is incorporated herein by reference). An external device can be used to wirelessly control the pump. The reservoir associated with the pump can be refillable via percutaneous injection.

A treatment regimen that includes at least one estrogen receptor modulator optionally in combination with one or more steroid hormones or metabolites, modulators, or analogs thereof for treatment of cardiovascular disease or condition in a subject can be delivered using an implantable delivery method that incorporates a MEMS (Micro Electro Mechanical Systems) fabricated microchip. Examples of MEMS and/or microfabricated devices for potential delivery of a therapeutic agent are described in U.S. Pat. Nos. 5,993,414; 6,454,759; and 6,808,522, which are incorporated herein by reference. The MEMS implantable delivery method can have one or more microfabricated drug reservoirs, for example, microparticle reservoirs, silicon microarray reservoirs, and/or polymer microreservoirs as described by Grayson, et al., Proceedings of the IEEE, 92: 6-21, 2004, which is incorporated herein by reference. Microparticles fabricated from silicon can be used that contain an internal space that is loaded with drug using a microinjector and capped, for example, with a slow dissolving gelatin or starch. Polymer microreservoirs can be fabricated by micromolding poly(dimethylsiloxane) or by patterning in multilayer poly(D-lactic acid) and (vinyl alcohol), for example. In some instances, the polymer microreservoirs can be capped with polymers that degrade at various rates in vivo depending upon the length of the polymer, allowing for controlled release of multiple doses.

Alternatively, an array of microreservoirs on a microchip can be used in which each dose of a treatment regimen including at least one estrogen receptor modulator and optionally including one or more steroid hormones, or metabolites, modulators, or analogs thereof is contained in its own reservoir and capped by an environmentally sensitive material. For example, the microreservoirs can be capped with a gold membrane that is weakened and ruptured by electrochemical dissolution in response to application of an anode voltage to the membrane in the presence of chloride ions, resulting in release of drug as described in U.S. Pat. No. 5,797,898 and in Prescott, et al., Nat. Biotech., 24:437-438, 2006, which are incorporated herein by reference. Alternatively, the microreservoirs can be capped by a temperature sensitive material that can be ruptured in response to selective application of heat to one or more of the reservoirs as described in U.S. Pat. No. 6,669,683, which is incorporated herein by reference. Wireless induction of a voltage or thermal trigger, for example, to a given reservoir of the microarray by a subject or other individual would enable on-demand release of one or more steroid hormones. Alternatively, the microchip array can incorporate a sensor component that signals release of one or more steroid hormones by a closed-loop mechanism in response to a chemical or physiological state as described in U.S. Pat. No. 6,976,982, which is incorporated herein by reference.

In some instances, the implantable delivery method can incorporate a natural and/or synthetic stimulus-responsive hydrogel or polymer that changes confirmation rapidly and reversibly in response to environmental stimuli, for example, temperature, pH, ionic strength, electrical potential, light, magnetic field or ultrasound (see, e.g., Stubbe, et al., Pharmaceutical Res., 21:1732-1740, 2004, which is incorporated herein by reference). Examples of polymers are described in U.S. Pat. Nos. 5,830,207; 6,720,402; and 7,033,571, which are incorporated herein by reference. In some instances, the at least one estrogen receptor modulator optionally in combination with one or more steroid hormones, or metabolites, modulators, or analogs thereof to be delivered by the implantable delivery method can be dissolved or dispersed in the hydrogel or polymer. Alternatively, a hydrogel and/or other stimulus-responsive polymer can be incorporated into an implantable delivery device. For example, a hydrogel or other polymer or other smart material can be used as an environmentally sensitive actuator to control flow of a therapeutic agent out of an implantable device as described in U.S. Pat. Nos. 6,416,495; 6,571,125; and 6,755,621, which are incorporated herein by reference. As such, an implantable delivery device can incorporate a hydrogel or other polymer that modulates delivery of at least one estrogen receptor modulator optionally in combination with one or more steroid hormones, or metabolites, modulators, or analogs thereof in response to environmental conditions.

In some instances, the implantable delivery method can be nonprogammable, delivering a predetermined dosage of at least one estrogen receptor modulator optionally in combination with one or more steroid hormones, or metabolites, modulators, or analogs thereof. For example, at least one estrogen receptor modulator optionally in combination with one or more steroid hormones can be administered using continuous infusion. Alternatively, the dosage of at least one estrogen receptor modulator optionally in combination with one or more steroid hormones can be predetermined to deliver a dose based on a timing mechanism associated with the implantable device. For example, the timing device can be linked to the menstrual cycle and the established baseline levels of estrogen, progesterone, and testosterone, for example. Alternatively, the implantable device can be programmable, having on/off and/or variable delivery rates based on either external or internal control. External control can be mediated by manual manipulation of a hand-operated pulsative pump with one-way valves associated with a delivery device implanted near the surface of the skin, for example. Alternatively, external control can be mediated by remote control through an electromagnetic wireless signal, for example, infrared or radio waves that are able to trigger an electrical stimulus within the implanted device. Examples of remote control drug delivery devices are described in U.S. Pat. Nos. 5,928,195; 6,454,759; and 6,551,235, which are incorporated herein by reference. As such, at least one estrogen receptor modulator optionally in combination with one or more steroid hormones, or metabolites, modulators, or analogs thereof can be delivered by continuous infusion in response to an “on” trigger and stopped in response to an “off” trigger, for example. Alternatively, at least one estrogen receptor modulator optionally in combination with one or more steroid hormones, or metabolites, modulators, or analogs thereof can be delivered as a microbolus, for example, in response to an “on” trigger as described in U.S. Pat. No. 6,554,822, which is incorporated herein by reference. External control can be initiated by a caregiver. Alternatively, a subject can initiate delivery of at least one estrogen receptor modulator optionally in combination with one or more steroid hormones. As such, the system can have a built in mechanism to limit the number of allowable doses by a subject and/or caregiver in a given time frame as described, for example, in U.S. Pat. No. 6,796,956, which is incorporated herein by reference.

An implantable device for delivery of a treatment regimen including at least one estrogen receptor modulator optionally in combination with one or more steroid hormones or metabolites, modulators, or analogs thereof can be powered by a standard lithium battery. In some instances, the battery can be rechargeable. For example, a battery associated with an implantable device can be recharged transcutaneously via inductive coupling from an external power source temporarily positioned on or near the surface of the skin as described in U.S. Pat. No. 7,286,880, which is incorporated herein by reference. Alternatively, the energy source for an implantable device can come from within the subject. For example, an implantable device can be powered by conversion of thermal energy from the subject into an electrical current as described in U.S. Pat. No. 7,340,304, which is incorporated herein by reference. Other methods of recharging or directly driving a battery associated with an implantable device can include, but are not limited to, electromagnetic energy transmission, piezoelectric power generation, thermoelectric devices, ultrasonic power motors, radio frequency recharging and optical recharging methods as described in Wei & Liu. Front. Energy Power Eng. China 2:1-13, 2008, which is incorporated herein by reference.

In some instances a treatment regimen including two or more estrogen receptor modulator optionally in combination with two or more steroid hormones, or metabolites, modulators, or analogs thereof can be administered using the same format. Alternatively, a combination of two or more modes of administration can be used for each dosing regimen. For example, a first estrogen receptor modulator optionally in combination with one or more steroid hormones or metabolite, modulator, or analog thereof can be provided by transdermal administration and the second steroid hormones or metabolite, modulator, or analog thereof can be provided by vaginal administration. As another example, the first estrogen receptor modulator can be provided by oral administration, the second estrogen receptor modulator optionally in combination with one or more steroid hormones or metabolite, modulator, or analog thereof can be provided by transdermal administration, and a third estrogen receptor modulator optionally in combination with one or more steroid hormones or metabolite, modulator, or analog thereof can be provided by transdermal administration.

Examples of compounds used as part of a treatment regimen can be administered by methods including, but not limited to, transdermal delivery, parenteral delivery, subcutaneous, intravenous, intramuscular, intraperitoneal, transdermal, transbuccal, intraocular, intravitreal, intravaginal, oral delivery, nasal delivery, e.g., by inhalation, intra-nasal spray, depot injections, implants, or infusions. Examples of compounds useful to treat a cardiovascular disease or condition and to maintain a physiological cyclic level of one or more steroid hormones can include, but are not limited to, natural and synthetic compounds, metabolites, modulators, or analogs thereof. Compounds that can be used as part of the treatment regimen include at least one selective estrogen receptor modulator (SERM). Examples of SERMs can include, but are not limited to, tamoxifen, idoxifene, toremifene and raloxifene. The selective estrogen receptor modulators can include, but are not limited to, at least one selective estrogen receptor α agonist and/or at least one selective estrogen receptor β agonist. The at least one selective estrogen receptor α agonist can include, but is not limited to, 17β-estradiol or propylpyrazole triol, 3,17-dihydroxy-19-nor-17α-pregna-1,3,5 (10)triene-21,16α-lactone. Proc. Natl. Acad. Sci. USA 101: 5129-5134, 2004, which is incorporated herein by reference. The at least one selective estrogen receptor β agonist can include, but is not limited to, diarylpropionitrile, ERB-041 [Harris et al., Endocrinology 144: 4241-4249, 2003], WAY-202196, WAY-214156 (2,8-dihydroxy-6H-dibenzo[c,h]chromene-4,12-dicarbonitrile), 8-vinylestra-1,3,5 (10)-triene-3,17β-diol, or a selective estrogen receptor modulator. Cvoro et al., J. Immunol., 180: 630-636, 2008; Proc. Natl. Acad. Sci. USA 101: 5129-5134, 2004, which is incorporated herein by reference.

Pharmaceutical compounds and compositions that can be used to alter estrogen levels, for example, can include, but are not limited to, natural compounds with estrogenic activity such as estradiol (estradiol-17β), estriol, estrone, and their metabolites such as 2-hydroxyestrone, 2-methoxyestrone, 16α-hydroxyestrone, 17α-estradiol, 2-hydroxy-estradiol-17β, 2-methoxyl-estradiol-17β 6β-hydroxyl-estradiol-17β, 3-sulfate, 3-glucuronide, and 16-glucuronide; synthetic steroidal compounds having estrogenic activity such as estradiol 17β-acetate, estradiol 17β-cypionate, estradiol 17β-propionate, estradiol 3-benzoate, ethinyl estradiol, piperazine estrone sulfate, mestranol, and quinestrol; synthetic non-steroidal compounds having estrogenic activity such as diethylstilbestrol, chlorotrianisene, and methallenestril; and plant derived phytoestrogens having estrogenic activity such as coumestrol, 4′methoxycoumestrol, repensol, trifoliol, daidzein, formononetin, genistein, and biochanin A. Esters, conjugates and prodrugs of suitable estrogens can also be used. Examples of estrogen prodrugs that can be used include, but are not limited to, estradiol acetate (which is converted in vivo to 17β-estradiol) and mestranol (which is converted in vivo to ethinyl estradiol). In some instances, a combination of estrogens can be used, see e.g. U.S. Pat. No. 6,911,438, which is incorporated herein by reference and provides a combination of three estrogens 2-hydroxyestrone, 17β-estradiol, and estriol, for example in a ratio determined by the method.

In some instances, the pharmaceutical compounds and compositions used to alter a hormone level, can include a natural precursor. For example, steroid hormone levels can be altered by providing a natural precursor, for example, testosterone, which can be converted in vivo to estradiol, or androstenedione, which can be converted to estrone or may be converted to testosterone. The treatment regimen can include a compound with enzymatic activity able to convert a naturally occurring precursor so as to alter a hormone level, for example a cytochrome P450 enzyme, or analog or modulator thereof. The treatment regimen can include modulating the activity of a resident enzyme, such as one active in steroidogenesis, by adding an inhibitor or activator.

Pharmaceutical compounds and compositions that can be used as part of a treatment regimen to alter progesterone levels, for example, can include, but are not limited to, natural and synthetic compounds having progestational activity, for example, progesterone, levonorgestrel, norethindrone, norethindrone acetate, desogestrel, gestodene, dienogest, norgestimate, cyproterone acetate, norelgestromin, etonogestrel, ethynodiol diacetate, norgestrel, trimegestone, medroxyprogesterone acetate, chlormadinone acetate, drospirenone, and other natural and/or synthetic gestagens. Esters, conjugates, and prodrugs of suitable progestins can also be used. Additional compounds can include metabolites and/or analogs of progesterone, for example, 20α-DH-P (4-pregnen-20α-ol-3-one), 5α-DH-P (5α-pregnan-3,20-dione), 3β,5α-TH-P (5α-pregnan-3b-ol-20-one), 20α-DH,5α-DH-P (5α-pregnan-20α-ol-3-one), 16α-OH-P (4-pregnen-16α-ol-3,20-dione), 5β-DH-P (5β-pregnan-3,20-dione), 20α-DH,3β,5α-TH-P (5α-pregnan-3β,20α-diol), 20α-DH, 3α,5α-TH-P (5α-pregnan-3α,20α-diol), 3α,5α-TH-P (5α-pregnan-3α-ol-20-one), 11α-OH-P (4-pregnen-11α-ol-3,20-dione), 11β-OH-P (4-pregnen-11β-ol-3,20-dione), 20α-DH,3α,5β-TH-P (5β-pregnan-3α,20α-diol), 17α-OH-P (4-pregnen-17α-ol-3-one), 17α-OH,20α-DH-P (4-pregnen-17,20α-diol-3-one) and 3α,5β-TH-P (5β-pregnan-3α-ol-20-one) (see, e.g., Quinkler, et al., Eur. J. Endocrinol. 146:789-800, 2002, which is incorporated herein by reference).

Pharmaceutical compounds and compositions that can be used as part of a treatment regimen to alter testosterone and androgen levels, for example, can include, but are not limited to, natural androgens and metabolites thereof such as testosterone, dihydrotestosterone (DHT), dehydroepiandrosterone (DHEA), dehydroepiandrosterone sulfate (DHEAS), androstenedione, androst-5-ene-3β,17β-diol; synthetic androgens such as testosterone undecanoate, testosterone propionate, testosterone cypionate, testosterone enanthate, methyltestosterone, fluoxymesterone, oxymetholone, oxandrolone, nandrolone decanoate.

A treatment regimen to alter levels of one or more steroid hormones can include compounds that stimulate the synthesis of one or more steroid hormones. Such compounds can include gonadotropin hormones, for example, luteinizing hormone (LH) and follicle stimulating hormone (FSH), that modulate testosterone, estrogen and progesterone levels during the menstrual cycle. Examples of purified follicle stimulating hormone can include, but are not limited to, urofollitropin (uFSH) purified from urine of postmenopausal women, recombinant forms of follicle stimulating hormone (rFSH) follitropin alfa and follitropin p. Examples of luteinizing hormone include recombinant human luteinizing hormone (rLH) lutropin.

### Pharmaceutical Formulations

The methods described herein for treating cardiovascular disease or condition in a mammalian subject in need thereof include an individualized treatment regimen for the subject. The individualized treatment regimen includes at least one estrogen receptor modulator optionally in combination with replacement therapy for one or more steroid hormones, metabolites, modulators, or analogs thereof. The treatment regimen can be based upon information derived from pre-menopausal hormone levels or pre-disease hormone levels in the subject and current physiologic hormone levels. A treatment regimen includes a pharmaceutical formulation of at least one estrogen receptor modulator optionally in combination with one or more steroid hormones, or metabolites, modulators, or analogs thereof for treating cardiovascular disease or condition and maintaining a substantially physiological level of one or more steroid hormones in a subject. The pharmaceutical formulation can be formulated neat or can be combined with one or more acceptable carriers, diluents, excipients, and/or vehicles, for example, buffers, surfactants, preservatives, solubilizing agents, isotonicity agents, and stabilizing agents as appropriate. A “pharmaceutically acceptable” carrier, for example, can be approved by a regulatory agency of the state and/or Federal government, for example, the United States Food and Drug Administration (US FDA) or listed in the U.S. Pharmacopeia or other generally recognized pharmacopeia for use in animals, and more particularly in humans. Conventional formulation techniques generally known to practitioners are described in Remington: The Science and Practice of Pharmacy, 20^{th} Edition, Lippincott Williams & White, Baltimore, Md. (2000), which is incorporated herein by reference.

Acceptable pharmaceutical carriers can include, but are not limited to, the following: sugars, such as lactose, glucose and sucrose; starches, such as corn starch and potato starch; cellulose, and its derivatives, such as sodium carboxymethyl cellulose, ethyl cellulose, cellulose acetate, and hydroxymethylcellulose; polyvinylpyrrolidone; cyclodextrin and amylose; powdered tragacanth; malt; gelatin, agar and pectin; talc; oils, such as mineral oil, polyhydroxyethoxylated castor oil, peanut oil, cottonseed oil, safflower oil, sesame oil, olive oil, corn oil and soybean oil; polysaccharides, such as alginic acid and acacia; fatty acids and fatty acid derivatives, such as stearic acid, magnesium and sodium stearate, fatty acid amines, pentaerythritol fatty acid esters; and fatty acid monoglycerides and diglycerides; glycols, such as propylene glycol; polyols, such as glycerin, sorbitol, mannitol and polyethylene glycol; esters, such as ethyl oleate and ethyl laurate; buffering agents, such as magnesium hydroxide, aluminum hydroxide and sodium benzoate/benzoic acid; water; isotonic saline; Ringer's solution; ethyl alcohol; phosphate buffer solutions; other non-toxic compatible substances employed in pharmaceutical compositions.

A treatment regimen including a pharmaceutical formulation of at least one estrogen receptor modulator optionally in combination with one or more steroid hormones or metabolites, modulators, or analogs thereof for treating cardiovascular disease or condition and maintaining a substantially physiological level of the one or more steroid hormones can be formulated in a pharmaceutically acceptable liquid carrier. The liquid carrier or vehicle can be a solvent or liquid dispersion medium comprising, for example, water, saline solution, ethanol, a polyol, vegetable oils, nontoxic glyceryl esters, and suitable mixtures thereof. The solubility of a chemical blocking agent can be enhanced using solubility enhancers, for example, water; diols, such as propylene glycol and glycerol; mono-alcohols, such as ethanol, propanol, and higher alcohols; DMSO (dimethylsulfoxide); dimethylformamide, N,N-dimethylacetamide; 2-pyrrolidone, N-(2-hydroxyethyl)pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones). The proper fluidity can be maintained, for example, by the formation of liposomes, by the maintenance of the necessary particle size in the case of dispersions or by the use of surfactants. One or more antimicrobial agent can be included in the formulation, for example, parabens, chlorobutanol, phenol, sorbic acid, and/or thimerosal to prevent microbial contamination. In some instances, it may be preferable to include isotonic agents, for example, sugars, buffers, sodium chloride or combinations thereof.

A treatment regimen including a pharmaceutical formulation of at least one estrogen receptor modulator optionally in combination with one or more steroid hormones or metabolites, modulators, or analogs thereof for treating cardiovascular disease or condition and maintaining a substantially physiological level of the one or more steroid hormones can be formulated for transdermal delivery. For example, water-insoluble, stratum corneum-lipid modifiers, for example 1,3-dioxanes, 1,3-dioxolanes and derivatives thereof, 5-, 6-, 7-, or 8-numbered lactams (e.g., butyrolactam, caprolactam), morpholine, cycloalkylene carbonate have been described for use in transdermal iontophoresis (see, e.g., U.S. Pat. No. 5,527,797, which is incorporated herein by reference). Other suitable penetration-enhancing agents can include but are not limited to ethanol, hexanol, cyclohexanol, polyethylene glycol monolaurate, azacycloalkan-2-ones, linoleic acid, capric acid, lauric acid, neodecanoic acid hexane, cyclohexane, isopropylbenzene; aldehydes and ketones such as cyclohexanone, acetamide; N,N-di(lower alkyl)acetamides such as N,N-diethylacetamide, N,N-dimethyl acetamide; N-(2-hydroxyethyl)acetamide; esters such as N,N-di-lower alkyl sulfoxides; essential oils such as propylene glycol, glycerine, isopropyl myristate, and ethyl oleate; salicylates; and mixtures of any of the above (see, e.g., U.S. Patent Publication 2008/0119449).

In some instances, the treatment regimen including a pharmaceutical formulation of at least one estrogen receptor modulator optionally in combination with one or more steroid hormones or metabolites, modulators, or analogs thereof for treating cardiovascular disease or condition and maintaining a substantially physiological level of the one or more steroid hormones can be formulated in a dispersed or dissolved form in a hydrogel or polymer associated with, for example, implantable or a transdermal delivery method. Examples of hydrogels and/or polymers can include, but are not limited to, gelled and/or cross-linked water swellable polyolefins, polycarbonates, polyesters, polyamides, polyethers, polyepoxides and polyurethanes, for example, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(allyl alcohol). Other suitable polymers can include, but are not limited to, cellulose ethers, methyl cellulose ethers, cellulose and hydroxylated cellulose, methyl cellulose and hydroxylated methyl cellulose, gums such as guar, locust, karaya, xanthan gelatin, and derivatives thereof. For iontophoresis, for example, the polymer or polymers can include an ionizable group, for example, (alkyl, aryl or aralkyl) carboxylic, phosphoric, glycolic or sulfonic acids, (alkyl, aryl or aralkyl) quaternary ammonium salts and protonated amines and/or other positively charged species as described in U.S. Pat. No. 5,558,633, which is incorporated herein by reference in its entirety.

Information regarding formulation of FDA approved steroid hormones, or metabolites, modulators, or analogs thereof can be found in the package insert and labeling documentation associated with each approved agent. A compendium of package inserts and FDA approved labeling can be found in the Physician's Desk Reference. Alternatively, formulation information for approved chemical blocking agents can be found on the internet at websites, for example, www.drugs.com and www.rxlist.com. For example, PREMARIN, an oral form of conjugated equine estrogens, contains active drug, calcium phosphate tribasic, hydroxypropyl cellulose, microcrystalline cellulose, powdered cellulose, hypromellose, lactose monohydrate, magnesium stearate, polyethylene glycol, sucrose, and titanium dioxide. For those steroid hormones or metabolites, modulators, or analogs thereof that do not currently have a formulation appropriate for use in any of the delivery methods described above, an appropriate formulation can be determined empirically and/or experimentally using standard practices. The pharmaceutical compositions are generally formulated as sterile, substantially isotonic and in full compliance with all Good Manufacturing Practice (GMP) regulations of the U.S. Food and Drug Administration.

### Kits

The invention provides kits comprising the compositions, e.g., nucleic acids, expression cassettes, vectors, cells, polypeptides (e.g., Scd1 polypeptides or toll-like receptor 2-signal activating polypeptides) and/or antibodies of the invention. The kits also can contain instructional material teaching the methodologies and uses of the invention, as described herein.

The methods and compositions are further described with reference to the following examples; however, it is to be understood that the methods and compositions are not limited to such examples.

### EXAMPLES

### Example 1

### Estrogen Receptor α Agonist Treatment in Female Subject with APOE ε2/ε3 Genetic Profile and a Cardiovascular Disease

A treatment regimen that includes an estrogen receptor modulator is designed to treat a female subject diagnosed with a cardiovascular disease. The treatment regimen is based on her genetic APOE allelic profile and her current and historic levels of steroid hormones. The female subject is diagnosed with a cardiovascular disease, e.g., coronary heart disease, by her primary care physician and cardiologist. The diagnosis of coronary heart disease or other possible cardiovascular disease is made based on a combination of electrocardiogram, stress testing, echocardiography, chest X-ray, blood tests, electron-beam computed tomography, coronary angiography, and/or cardiac catheterization. The medical examination of the female subject includes APOE allelic profiling to determine the presence of ε2, ε3, and/or ε4 alleles in the female subject. The presence of certain alleles in the APOE profile is correlated with the risk for developing a cardiovascular disease. In particular the APOE ε4 allele is strongly associated with cardiovascular disease in perimenopausal and postmenopausal women. As part of the female subject's annual medical examination, the current steroid hormone levels of the female subject are assayed and compared to steroid hormone levels measured prior to disease diagnosis.

The APOE allelic profile of the female subject is determined using polymerase chain reaction (PCR) amplification of genomic DNA derived from the female subject's whole blood. Briefly, blood is drawn from the female subject using standard phlebotomy methods into an anti-coagulant solution (e.g., trisodium citrate, heparin, or EDTA). Genomic DNA is extracted from the whole blood of the female subject using detergent, proteinase K, phenol/chloroform extraction, and ammonium acetate/ethanol precipitation. PCR amplification is performed using the extracted genomic DNA and oligonucleotide primers specific to a 244 base-pair fragment within exon 4 of the APOE gene as outlined in Hixon, et al., J. Lipid Res. 31: 545-548, 1990, which is incorporated herein by reference. See, e.g., Hegele. Clin. Chem. 45: 1579-1580, 1999; and Appel, et al., Clin. Chem. 41:187-190, 1995, which are incorporated herein by reference. The PCR fragment containing a portion of the APOE gene of the female subject is digested with the restriction enzyme HhaI and chromatographed on a 10% polyacrylamide gel in Tris-borate-EDTA buffer. The resulting restriction map banding pattern creates a characteristic pattern of DNA bands for each of the common APOE alleles (ε2, ε3, and ε4) present in the female subject's genomic DNA. Based on the results of this analysis, the female subject is determined to have a banding pattern consistent with an allelic profile for ε2/ε3. The information regarding the female subject's APOE allelic profile is entered into the subject's medical record.

The steroid hormone levels in the whole blood of the female subject are assessed by one or more biological assays on a yearly, quarterly, or monthly basis before diagnosis and after diagnosis of coronary heart disease. The steroid hormone levels of the female subject are assessed using a number of techniques including immunoassay, gas or liquid chromatography, mass spectrometry, a recombinant cell based assay, assay arrays, microfluidic devices, or a combination hereof. For an immunoassay, a competitive enzyme-linked immunosorbent assay (ELISA) is used in which the steroid hormone in the blood sample competes for binding to a steroid hormone specific antibody with a known amount of labeled steroid hormone standard. The steroid hormone specific antibody can be immobilized on a substrate, e.g., the surface of a microtiter plate. The amount of labeled steroid hormone standard bound to the immobilized antibody is inversely proportional to the amount of steroid hormone in the blood sample and is determined by chromogenic, fluorogenic, chemiluminescent, or radioactive methods, depending upon the label associated with the standard. ELISA kits designed to measure steroid hormones are commercially available for estradiol, estrone, estriol, testosterone, DHEA, progesterone, follicle stimulating hormone, luteinizing hormone (from, e.g., Cayman Chemical, Ann Arbor, Mich.; Calbiotech, Spring Valley, Calif.; Beckman Coulter, Fullerton, Calif.). Estradiol, for example, is measured in isolated serum or EDTA treated plasma from the female subject using an Estradiol Enzyme Immunoassay Test Kit (cat #11110, Oxis International, Inc. Foster City) in which the estradiol standard is labeled with horseradish peroxidase and color detection is performed using TMB reagent (3,3′, 5,5′ tetramethylbenzidine). Similar assays are performed to assess the levels of other steroid hormones. Data regarding the steroid hormone levels of a female subject are entered into the subject's medical records.

The steroid hormone levels of the female subject assessed as part of the current medical work-up following diagnosis of coronary heart disease are compared with the historic steroid hormone levels of the subject measured at different time points prior to the current disease diagnosis. Data regarding the historic steroid hormone levels of a female subject are part of her medical record. The female subject's physician maintains an ongoing medical history containing all or part of the subject's medical records that serves to monitor changes in the hormone levels in the female subject. In the present case, the female subject's historical estrogen levels recorded several years before diagnosis and at a time during her entry into perimenopause are about 50 pg/ml, comparable to other women entering perimenopause. The female subject's current hormone levels are well below 50 pg/ml, consistent with postmenopause. Upon considering the current and historic medical history of the female subject, the physician designs an individualized treatment regimen for the female subject based on her APOE allelic profile and her current and historic steroid hormone levels.

Based on the APOE ε2/ε3 profile of the female subject, her current hormone levels, and her past hormone levels prior to the onset and after the onset of coronary heart disease, a treatment regimen is prescribed by her physician that includes an estrogen receptor α agonist taken daily over a period of time. In this example, the physician prescribes the estrogen receptor α agonist, 17β-estradiol. 17β-estradiol is available in multiple forms including oral tablet (e.g., Estrace® or Progynova® oral 17β-estradiol), transdermal patch (e.g., Estraderm®, Alora®, Climara®, or Menostar™ transdermal 17β-estradiol), topical cream (e.g., Estrasorb™, EstroGel®, or Elestrin™ topical 17β-estradiol), and vaginal ring (e.g., Estring® vaginal 17β-estradiol). The female subject and her physician decide which of these forms of estradiol is appropriate for the subject based on ease of use and other medications that the subject is taking. For oral treatment with estradiol, the female subject is started on a once daily treatment regimen of Estrace® (0.5 mg tablet per 50 kg body weight of the subject). After 3 to 6 months of treatment, the female subject is reassessed for symptoms and pathology associated with coronary heart disease and for current levels of steroid hormones. If warranted, the dosage of 17β-estradiol is adjusted with 0.5 mg, 1.0 mg or 2.0 mg tablets up to a level of 2.0 mg per day per 50 kg body weight of the subject.

### Example 2

### Estrogen Receptor α Agonist Treatment in Female Subject with APOE ε2/ε3 Genetic Profile and Cardiovascular Disease

A treatment regimen that includes an estrogen receptor modulator is designed to treat a female subject diagnosed with a cardiovascular disease. The treatment regimen is based on her genetic APOE allelic profile and her current and historic levels of steroid hormones. The female subject is diagnosed with a cardiovascular disease, e.g., coronary heart disease, by her primary care physician and cardiologist. The diagnosis of coronary heart disease as well as other cardiovascular diseases is made based on a combination of electrocardiogram, stress testing, echocardiography, chest X-ray, blood tests, electron-beam computed tomography, coronary angiography, and/or cardiac catheterization. The medical examination of the female subject includes APOE allelic profiling to determine the presence of ε2, ε3, and ε4 alleles. The presence of certain APOE alleles in the APOE allelic profile is correlated with the risk for developing a cardiovascular disease. In particular the apo ε4 allele is strongly associated with cardiovascular disease in perimenopausal and postmenopausal women. As part of the female subject's annual medical examination, the current steroid hormone levels of the female subject are assayed and compared to steroid hormone levels measured prior to disease diagnosis.

The APOE allelic profile of the female subject is determined using PCR amplification of genomic DNA derived from the subject's whole blood. Briefly, blood is drawn from the female subject using standard phlebotomy methods into an anti-coagulant solution (e.g., trisodium citrate, heparin, or EDTA). Genomic DNA is extracted from the whole blood of the female subject using any of a number of commercially available kits (e.g., GeneCatcher™ gDNA Automated Blood Kit, Invitrogen, Carlsbad, Calif.). PCR amplification using APOE specific primers, HhaI enzyme restriction mapping, and chromatography are performed as described in Example 1. The resulting restriction map banding pattern is indicative of the APOE allelic profile present in the female subject's genomic DNA. Based on the results of this analysis, the female subject is determined to have a banding pattern consistent with an ε2/ε3 allelic profile. The information regarding the female subject's APOE allelic profile is entered into the subject's medical record. See, e.g., Hixon, et al., J. Lipid. Res. 31:545-548, 1990, which is incorporated herein by reference.

The steroid hormone levels in the whole blood of the female subject are assessed by one or more biological assays on a yearly, quarterly, or monthly basis before diagnosis and after diagnosis of coronary heart disease. The steroid hormone levels of the female subject are assessed using a number of techniques including immunoassay, gas or liquid chromatography, mass spectrometry, a recombinant cell based assay, assay arrays, microfluidic devices, or a combination hereof. Multiple steroid hormones including estradiol, estrone, estriol, 16-hydroxyestrone, and aldosterone, are assayed in the female subject's serum samples using a combination of liquid chromatography, electrospray ionization and mass spectrometry (LC-ESI-MS/MS; see, e.g., Guo, et al., Clin. Biochem. 41:736-741, 2008, which is incorporated herein by reference). The female subject's serum sample is deproteinized by extraction with acetonitrile followed by centrifugation at 13,000 rpm for 10 minutes. The supernatant is then loaded directly into the LC-ESI-MS/MS system where the samples are chromatographed. Standards are used to determine the elution profile of each steroid hormone and the respective peaks are submitted to electrospray ionization followed by mass spectrometry. Known quantities of a given steroid hormone are subjected to the same process and used to generate a standard curve against which the measured levels of hormone in the female subject's serum sample are compared. Similar assays are performed to assess the levels of other steroid hormones. Data regarding the steroid hormone levels of a female subject are entered into the subject's medical records.

The steroid hormone levels of the female subject assessed as part of the current medical examination following diagnosis of coronary heart disease are compared with the historic steroid hormone levels of the subject measured at different time points prior to the current disease diagnosis. Data regarding the historic steroid hormone levels of a female subject are part of her medical record as monitored by the physician. In the present case, the female subject's historical estrogen levels recorded several years before diagnosis and at a time prior to her entry into perimenopause are about 75 pg/ml, comparable to other premenopausal women. The female subject's current hormone levels are about 50 pg/ml, consistent with other perimenopausal women. The physician uses this data to design an individualized treatment regimen for the female subject based on her APOE allelic profile and her current and historic steroid hormone levels.

Based on the apoE2/E3 allelic profile of the female subject, her current hormone levels, and her past hormone levels prior to the onset and after the onset of coronary heart disease, a treatment regimen is prescribed by her physician that includes an estrogen receptor α agonist taken daily over a period of time. In this example, the physician prescribes the estrogen receptor α agonist, propylpyrazole triol (4,4′,4″-(4-propyl-1H-pyrazole-1,3,5-triyl)tris-phenol; PPT). PPT is formulated in a unit dosage form such as a pill or tablet. Each unit dosage contains from about 0.5 mg to about 150 mg, more usually about 0.1 mg to about 10 mg, of PPT per 50 kg body weight of the female subject and is administered one or more times per day as described by Katzenellenbogen, et al., in WO/2000/019994, which is incorporated herein by reference. For oral treatment with PPT, the female subject is started on at least once daily dosing with 10 mg PPT per 50 kg body weight of the female subject. After 3 to 6 months of treatment, the female subject is reassessed for symptoms and pathology associated with coronary heart disease and for current levels of steroid hormones. If warranted, the dosage of PPT is adjusted by the physician up to 150 mg per 50 kg body weight of the female subject administered at least once daily.

### Example 3

### Estrogen Receptor-β Agonist Treatment in Female Subject with APOE ε4 Allelic Profile and Cardiovascular Disease

A treatment regimen that includes an estrogen receptor modulator is designed to treat a female subject diagnosed with a cardiovascular disease. The treatment regimen is based on her genetic APOE allelic profile and her current and historic levels of steroid hormones. The female subject is diagnosed with a cardiovascular disease, e.g., coronary heart disease, by her primary care physician and cardiologist. The diagnosis of coronary heart disease or other cardiovascular diseases is made based on a variety of diagnostic tests as outlined in Example 1. As part of the medical examination, the genetic APOE allelic profile is performed to determine the presence of ε2, ε3, and/or ε4 alleles. In addition, the current steroid hormone levels of the female subject are determined. The current steroid hormone levels of the female subject are compared to steroid hormone levels measured prior to disease diagnosis as part of the female subject's annual or more frequent medical examinations.

The APOE allelic profile of the female subject is determined using PCR amplification of genomic DNA derived from the subject's whole blood. Briefly, blood is drawn from the female subject using standard phlebotomy methods into an anti-coagulant solution (e.g., trisodium citrate, heparin, or EDTA). Genomic DNA is extracted as described herein. PCR amplification using APOE specific primers, HhaI enzyme restriction mapping, and gel chromatography are performed as described in Example 1. The resulting restriction map banding pattern is indicative of the APOE allelic profile present in the female subject's genomic DNA. Based on the results of this analysis, the female subject is determined to have a banding pattern consistent with an ε4 allelic profile. The information regarding the female subject's APOE allelic profile is entered into the subject's medical record. See, e.g., Hixon, et al., J. Lipid. Res. 31:545-548, 1990, which is incorporated herein by reference.

The steroid hormone levels in the whole blood of the female subject are assessed by one or more biological assays on a yearly, quarterly, or monthly basis before diagnosis and after diagnosis of coronary heart disease. The steroid hormone levels of the female subject are assessed using a number of techniques including immunoassay, gas or liquid chromatography, mass spectrometry, a recombinant cell based assay, assay arrays, microfluidic devices, or a combination thereof as described herein. Data regarding the steroid hormone levels of a female subject are entered into the subject's medical records.

The steroid hormone levels of the female subject assessed as part of the medical examination following diagnosis of coronary heart disease are compared with the historic steroid hormone levels of the subject measured at different time points prior to the current disease diagnosis. Data regarding the historic steroid hormone levels of a female subject are part of her medical record. In the present case, the female subject's historical estrogen levels recorded several years before diagnosis and at a time prior to her entry into perimenopause are about 75 pg/ml, comparable to other premenopausal women. The female subject's current hormone levels are about 50 pg/ml, consistent with other perimenopausal women. The physician uses this information to design an individualized treatment regimen for the female subject based on her APOE allelic profile and her current and historic estrogen levels.

Based on the APOE ε4 allelic profile of the female subject, her current hormone levels, and her past hormone levels prior to the onset and after the onset of coronary heart disease, a treatment regimen is prescribed by her physician that includes an estrogen receptor β agonist taken daily over a period of time. In this example, the physician prescribes the estrogen receptor β agonist, 2-(3-fluoro-4-hydroxyphenyl)-7-vinyl-1,3-benzoxazol-5-ol (ERB-041). ERB-041 is formulated in a unit dosage form that is a capsule. Each capsule contains from about 1 mg to about 125 mg per 50 kg body weight of the female subject as described by Rowley, et al., U.S. Patent Application 2006/0121109, which is incorporated herein by reference. Each capsule is administered one or more times per day. For oral treatment with ERB-041, the female subject is started on at least once daily dosing with 10 mg ERB-041 per 50 kg body weight of the female subject. After 3 to 6 months of treatment, the female subject is reassessed for symptoms and pathology associated with coronary heart disease and for current levels of steroid hormones. If warranted, the dosage of ERB-041 is adjusted by the physician up to 125 mg per 50 kg body weight of the female subject administered at least once daily.

### Example 4

### Treatment Regimen Based on Computer-Accessible Medical History of the Female Subject for Treatment of a Female Subject with Cardiovascular Disease

A treatment regimen that includes an estrogen receptor modulator and replacement therapy including one or more steroid hormones or metabolites or modulators thereof is designed to treat a female subject diagnosed with a cardiovascular disease. The treatment regimen is based on her genetic APOE allelic profile and her current and historic levels of steroid hormones. The female subject is diagnosed with a cardiovascular disease, e.g., atherosclerosis, by her primary care physician and cardiologist. The diagnosis of atherosclerosis as well as other cardiovascular diseases is made based on a combination of blood pressure changes, coronary angiography, electron beam computed tomography, stress testing, echocardiography. The medical examination of the female subject includes APOE allelic profiling to determine the presence of APOE ε2, ε3, and/or ε4 alleles. The presence of certain APOE alleles in the APOE allelic profile is correlated with the risk for developing a cardiovascular disease. In particular the ε4 allele is strongly associated with cardiovascular disease in perimenopausal and postmenopausal women. As part of the female subject's annual medical examination, the current steroid hormone levels of the female subject are assayed and compared to steroid hormone levels measured prior to disease diagnosis.

The APOE allelic profile of the female subject is determined using polymerase chain reaction (PCR) amplification of genomic DNA derived from the subject's whole blood. Briefly, blood is drawn from the female subject using standard phlebotomy methods into an anti-coagulant solution (e.g., trisodium citrate, heparin, or EDTA). Genomic DNA is extracted from the whole blood of the female subject using any of a number of commercially available kits (e.g., GeneCatcher™ gDNA Automated Blood Kit, Invitrogen, Carlsbad, Calif.). PCR amplification is performed using the extracted genomic DNA and oligonucleotide primers specific to a 244 base-pair fragment within exon 4 of the APOE gene as outlined in Hixon, et al., J. Lipid Res. 31:545-548, 1990, which is incorporated herein by reference. The PCR fragment containing a portion of the APOE gene is digested with the restriction enzyme HhaI and chromatographed on a 10% polyacrylamide gel in Tris-borate-EDTA buffer. The resulting restriction map banding pattern creates a characteristic pattern of DNA bands for each of the common APOE alleles (ε2, ε3, or ε4) present in the female subject's genomic DNA. Based on the results of this analysis, the female subject is determined to have a banding pattern consistent with an ε2/ε3 allelic profile. See, e.g., Hegele. Clin. Chem. 45: 1579-1580, 1999; and Appel, et al., Clin. Chem. 41:187-190, 1995, which are incorporated herein by reference.

The steroid hormone levels in the whole blood of the female subject are assessed by one or more biological assays on a yearly, quarterly, or monthly basis before diagnosis and after diagnosis of atherosclerosis. Routine testing of steroid hormone levels is part of the female subject's annual physical examination. A baseline measurement of steroid hormone levels to establish a physiological level in the subject is preferably made at a time prior to the female subject's entry into perimenopause. Preferably, the female subject is not using hormone-based contraceptives at the time of testing as these can complicate the measurement of endogenous levels of hormones. If the female subject is using hormone-based contraceptives, she is asked to stop using this form of contraception and use an alternative, non-hormonal form of contraception until testing is complete. Testing is initiated following one or more normal menses when endogenous hormones are presumed to have returned to normal levels.

The steroid hormone levels of the female subject are assessed using a number of techniques including immunoassay, gas or liquid chromatography, mass spectrometry, a recombinant cell based assay, assay arrays, microfluidic devices, or a combination thereof. Using a cell-based bioassay, the levels of one or more steroid hormones in a bodily fluid (e.g., blood) of the female subject are assayed using a yeast strain (e.g., Saccharomyces cerevisiae) modified to express a recombinant hormone receptor that emits a measurable readout in response to binding a steroid hormone. In this cell-based bioassay, the yeast are transformed with the human estrogen receptor and an estrogen response element (ERE) upstream of the yeast iso-1-cytochrome C promoter fused to the structural gene for β-galactosidase as described by Klein, et al., J. Clin. Endocrinol. Metab. 80: 2658-2660, 1995, which is incorporated herein by reference. Increased β-galactosidase activity in response to the presence of estrogen in the assay is assessed by calorimetric detection as measured using a spectrophotometer. A standard curve of known estrogen concentrations is generated and used to estimate the amount of endogenous estrogen in the blood of the female subject. Similar assays are performed to assess the levels of other steroid hormones. Data regarding the steroid hormone levels of a female subject are entered into the subject's medical records to document levels of steroid hormone in the female subject currently and in the past.

The female subject's physician compares the subject's current physiological steroid hormone levels with her historical steroid hormone levels and the subject's APOE profile as part of a current medical work-up following diagnosis of atherosclerosis in the female subject. The physician uses this information to design a treatment regimen to treat atherosclerosis and to maintain a substantially physiological level of one or more steroid hormone. Data regarding the historic steroid hormone levels of a female subject are part of her medical record. The physician utilizes a computer system including a computer program accessing a database that contains all or part of the subject's medical records and is used to monitor changes in the hormone levels based upon historic steroid hormone levels and current steroid hormone levels in the female subject and the APOE allelic profile of the female subject. In the present case, the female subject's historical estrogen levels measured several years before diagnosis and at a time during her entry into perimenopause are about 50 pg/ml, comparable to other women entering perimenopause. The female subject's current hormone levels are well below 50 pg/ml, consistent with postmenopause. The results of computational analysis are used by the physician to design an individualized treatment regimen for the female subject based on her APOE allelic profile and her current and historic estrogen, progestogen. and leutinizing hormone levels.

Based on the apoE2/E3 allelic profile of the female subject, her current hormone levels, and her past hormone levels prior to the onset and after the onset of atherosclerosis, a treatment regimen is prescribed by her physician that includes an estrogen receptor α agonist in combination with replacement therapy. In this example, the physician prescribes the estrogen receptor α agonist, propylpyrazole triol (PPT), in combination with the replacement therapy including 17β-estradiol either with or without added progestin. The female subject is started on at least once daily dosing with 10 mg PPT per 50 kg body weight of the subject based on described unit dosages of PPT ranging from about 0.5 mg to about 150 mg per 50 kg body weight of the subject. See, e.g., Katzenellenbogen, et al., in WO/2000/019994, which is incorporated herein by reference.

In addition, the physician compares the levels of one or more steroid hormones of the female subject before and after diagnosis of atherosclerosis and prescribes a level of replacement therapy designed to substantially return her steroid hormone levels to a physiological level. The appropriate physiological level of estrogen, for example, is a level measured prior to the diagnosis of atherosclerosis and is the level noted at premenopause (approximately 70 ng/ml) or perimenopause (approximately 50 ng/ml). The dose of estrogen in the replacement therapy is such that the amount of exogenous and endogenous estrogen brings the overall serum concentration of estrogen of the female subject back to a baseline physiological concentration established during premenopause, perimenopause and/or before the diagnosis of atherosclerosis. The female subject is started on a low dose of once daily 17β-estradiol (e.g., Estrace® oral 17β-estradiol; 0.5 mg tablet) in combination with a low dose of once daily medroxyprogesterone acetate (pregn-4-ene-3,20-dione, 17-(acetyloxy)-6-methyl-, (6a)-; e.g., Provera® oral medroxyprogesterone acetate; 2.5 mg tablet). Alternatively, the female subject is given the option of taking a single pill that contains both an estrogen and a progestogen (e.g., PREMPRO®. PREMPHASE® estrogen/progestin). After 3 to 6 months of treatment, the female subject is reassessed for symptoms and pathology associated with atherosclerosis and for current levels of steroid hormones. If warranted, the dosages of PPT and the replacement therapy are adjusted by the physician up to 150 mg PPT per 50 kg body weight of the subject, up to 2 mg 17β-estradiol and up to 10 mg medroxyprogesterone acetate per 50 kg body weight of the subject administered at least once daily dosing.

### Example 5

### Estrogen Receptor β Agonist in Combination with Replacement Therapy Including One or More Steroid Hormones for Treatment of a Female Subject with APOE ε4 Profile and Cardiovascular Disease

A treatment regimen that includes an estrogen receptor modulator and replacement therapy including one or more steroid hormones or metabolites or modulators thereof is designed to treat a female subject diagnosed with a cardiovascular disease. The treatment regimen is based on her genetic APOE allelic profile and her current and historic levels of steroid hormones. The female subject is diagnosed with a cardiovascular disease, e.g., atherosclerosis, by her primary care physician and cardiologist. The diagnosis of atherosclerosis as well as other cardiovascular diseases is made based on a combination of blood pressure changes, coronary angiography, electron beam computed tomography, stress testing, echocardiography. The medical examination of the female subject includes APOE allelic profiling to determine the presence of ε2, ε3, and/or ε4 alleles. The presence of certain APOE alleles in the APOE allelic profile is correlated with the risk for developing a cardiovascular disease. In particular the APOE ε4 allele is strongly associated with cardiovascular disease in perimenopausal and postmenopausal women. As part of the female subject's annual medical examination, the current steroid hormone levels of the female subject are assayed and compared to steroid hormone levels measured prior to disease diagnosis.

The APOE allelic profile of the female subject is determined using polymerase chain reaction (PCR) amplification of genomic DNA derived from the subject's whole blood. Briefly, blood is drawn from the female subject using standard phlebotomy methods into an anti-coagulant solution (e.g., trisodium citrate, heparin, or EDTA). Genomic DNA is extracted from whole blood of the female subject using detergent, proteinase K, phenol/chloroform extraction, and ammonium acetate/ethanol precipitation. PCR amplification is performed using the extracted genomic DNA and oligonucleotide primers specific to a 244 base-pair fragment within exon 4 of the APOE gene as outlined in Hixon, et al., J. Lipid Res. 31:545-548, 1990, which is incorporated herein by reference. The PCR fragment containing a portion of the APOE gene is digested with the restriction enzyme HhaI and chromatographed on a 10% polyacrylamide gel in Tris-borate-EDTA buffer. The resulting restriction map banding pattern creates a characteristic pattern of DNA bands for each of the common APOE alleles (ε2, ε3, and/or ε4) present in the female subject's genomic DNA. Based on the results of this analysis, the female subject is determined to have a banding pattern consistent with an ε4 allelic profile. See, e.g., Hegele. Clin. Chem. 45:1579-1580, 1999; and Appel, et al., Clin. Chem. 41:187-190, 1995, which are incorporated herein by reference.

The steroid hormone levels in the whole blood of the female subject are assessed by one or more biological assays on a yearly, quarterly, or monthly basis before diagnosis and after diagnosis of atherosclerosis. The steroid hormone levels of the female subject are assessed using a number of techniques including immunoassay, gas or liquid chromatography, mass spectrometry, a recombinant cell based assay, assay arrays, microfluidic devices, or a combination hereof. For an immunoassay, a competitive enzyme-linked immunosorbent assay (ELISA) is used in which the steroid hormone in the blood sample competes for binding to a steroid hormone specific antibody with a known amount of labeled steroid hormone standard. The steroid hormone specific antibody can be immobilized on a substrate, e.g., the surface of a microtiter plate. The amount of labeled steroid hormone standard bound to the immobilized antibody is inversely proportional to the amount of steroid hormone in the blood sample and is determined by chromogenic, fluorogenic, chemiluminescent, or radioactive methods, depending upon the label associated with the standard. ELISA kits designed to measure steroid hormones are commercially available for estradiol, estrone, estriol, testosterone, DHEA, progesterone, follicle stimulating hormone, luteinizing hormone (from, e.g., Cayman Chemical, Ann Arbor, Mich.; Calbiotech, Spring Valley, Calif.; Beckman Coulter, Fullerton, Calif.). Estradiol, for example, is measured in isolated serum or EDTA treated plasma from the female subject using an Estradiol Enzyme Immunoassay Test Kit (cat #11110, Oxis International, Inc. Foster City) in which the estradiol standard is labeled with horseradish peroxidase and color detection is performed using TMB reagent (3,3′,5,5′ tetramethylbenzidine). Similar assays are performed to assess the levels of other steroid hormones. Data regarding the steroid hormone levels of a female subject are entered into the subject's medical records.

The steroid hormone levels of the female subject assessed as part of the medical examination following diagnosis of atherosclerosis are compared with the historic steroid hormone levels of the subject measured at different time points prior to the current disease diagnosis. Data regarding the historic steroid hormone levels of a female subject are part of her medical record. A computer program containing all or part of the subject's medical records is used to monitor changes in the hormone levels. In the present case, the female subject's historical estrogen levels recorded several years before diagnosis and at an age consistent with perimenopause are about 50 pg/ml, comparable to other women experiencing perimenopause. The female subject's current hormone levels are well below 50 pg/ml, consistent with postmenopause. Computational analysis is used by the physician to design an individualized treatment regimen for the female subject based on her APOE allelic profile and her current and historic estrogen levels.

Based on the APOE ε4 allelic profile of the female subject, her current hormone levels, and her past hormone levels prior to the onset and after the onset of atherosclerosis, a treatment regimen is prescribed by her physician that includes an estrogen receptor β agonist in combination with replacement therapy. In this example, the physician prescribes the estrogen receptor β agonist, 2-(3-fluoro-4-hydroxyphenyl)-7-vinyl-1,3-benzoxazol-5-ol (ERB-041), and the replacement therapy includes 17β-estradiol either with or without added progestin. ERB-041 is formulated in a unit dosage form that is a capsule. Each capsule contains from about 1 mg to about 125 mg per 50 kg body weight of the female subject as described by Rowley, et al., U.S. Patent Application 2006/0121109, which is incorporated herein by reference. For oral treatment with ERB-041, the female subject is started on at least once daily dosing with 10 mg ERB-041 per 50 kg body weight of the female subject.

In addition, the physician compares the levels of one or more steroid hormones of the female subject before and after diagnosis of atherosclerosis and prescribes a level of replacement therapy designed to substantially return her steroid hormone levels to a physiological level. The appropriate physiological level of estrogen, for example, is a level measured prior to the diagnosis of atherosclerosis and is the level noted at premenopause (approximately 70 ng/ml) or perimenopause (approximately 50 ng/ml). The dose of estrogen in the replacement therapy is such that the amount of exogenous and endogenous estrogen brings the overall serum concentration of estrogen of the female subject back to a baseline physiological concentration established during premenopause, perimenopause and/or before the diagnosis of atherosclerosis. The female subject is started on a low dose of once daily 17β-estradiol (e.g., Estrace® oral 17β-estradiol; 0.5 mg tablet) in combination with a low dose of once daily medroxyprogesterone acetate (pregn-4-ene-3,20-dione, 17-(acetyloxy)-6-methyl-, (6a)-; e.g., Provera® oral medroxyprogesterone acetate; 2.5 mg tablet). Alternatively, the female subject is given the option of using a single pill, cream, vaginal ring, or transdermal patch that contains a combination of progestin derivative and conjugated estrogen or estradiol (e.g., PREMPRO®, PREMPHASE®, or Climara Pro® estrogen/progestin). After 3 to 6 months of treatment, the female subject is reassessed for symptoms and pathology associated with atherosclerosis and the current levels of steroid hormones in the subject. If warranted, the dose of ERB-041 and the does of replacement therapy are adjusted by the physician up to 125 mg ERB-041, up to 2 mg 17β-estradiol and up to 10 mg medroxyprogesterone acetate per 50 kg body weight of the subject administered at least once daily dosing.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

The state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Optical aspects of implementations will typically employ optically-oriented hardware, software, and or firmware.

In a general sense the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art after reading the disclosure herein. The various aspects and embodiments disclosed herein are for purposes of illustration and are not intended to be limiting, with the true scope and spirit being indicated by the following claims.

What is claimed is:

1.-34. (canceled)

35. A system comprising:
a signal-bearing medium including,
one or more instructions for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition.

36. The system of claim 35, wherein the mammalian subject's APOE allelic profile is at least one of APOE ε2 positive and APOE ε3 positive.

37. The system of claim 36, wherein the at least one estrogen receptor modulator includes at least one selective estrogen receptor α agonist.

38. The system of claim 36, wherein the mammalian subject's APOE allelic profile is ε4 negative.

39. The system of claim 35, further including one or more instructions for determining the at least one treatment regimen including at least one replacement therapy for one or more steroid hormones or metabolites or modulators thereof.

40. The system of claim 39, wherein the at least one treatment regimen including the least one replacement therapy is configured to increase levels of one or more of an estrogen and a progestogen, or metabolites or modulators thereof.

41. The system of claim 37, wherein the at least one selective estrogen receptor α agonist includes 17β-estradiol, propylpyrazole triol, or 3,17-dihydroxy-19-nor-17α-pregna-1,3,5 (10)triene-21,16α-lactone.

42. The system of claim 35, wherein the mammalian subject's APOE allelic profile is ε4 positive.

43. The system of claim 42, wherein the at least one estrogen receptor modulator includes at least one selective estrogen receptor β agonist.

44. The system of claim 39, wherein the at least one treatment regimen includes the at least one replacement therapy is configured to increase levels of one or more of an estrogen and a progestogen, or metabolites or modulators thereof.

45. The system of claim 44, wherein the at least one treatment regimen includes replacement therapy with one or more of an estrogen and a progestogen.

46. The system of claim 43, wherein the at least one selective estrogen receptor β agonist includes diarylpropionitrile, ERB-041, WAY-202196, WAY-214156 (2,8-dihydroxy-6H-dibenzo[c,h]chromene-4,12-dicarbonitrile), or 8-vinylestra-1,3,5 (10)-triene-3,17β-diol.

47. The system of claim 35, wherein the at least one estrogen receptor modulator is configured to maintain the mammalian subject's one or more steroid hormones or metabolites or modulators thereof at substantially physiological cyclic levels, and the at least one estrogen receptor modulator is in an amount effective to reduce cardiovascular disease or condition or alleviate symptoms thereof in the mammalian subject.

48. The system of claim 35, wherein the mammalian subject is female.

49. The system of claim 47, wherein the mammalian subject is perimenopausal or postmenopausal.

50. The system of claim 47, wherein the at least one treatment regimen is determined at least in part based on the premenopausal cyclic steroid hormone levels in the mammalian subject and on current cyclic steroid hormone levels in the mammalian subject.

51. The system of claim 35, wherein the mammalian subject is male.

52. The system of claim 35, further including one or more instructions for inputting information associated with the steroid hormone levels prior to disease diagnosis and on current steroid hormone levels in the mammalian subject.

53. The system of claim 35, wherein the at least one treatment regimen is configured to maintain a substantially physiological cyclic level of one or more steroid hormones prior to disease diagnosis in the mammalian subject in need thereof, and the at least one treatment regimen is in an amount effective to reduce cardiovascular disease or condition or alleviate symptoms thereof in the mammalian subject.

54. The system of claim 35, wherein the signal bearing medium includes a computer readable medium.

55. The system of claim 35, wherein the signal bearing medium includes a recordable medium.

56. The system of claim 35, wherein the signal bearing medium includes a communications medium.

57. A device comprising:
a system including a signal-bearing medium including,
one or more instructions for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to disease diagnosis.

58. The device of claim 57, wherein the mammalian subject's APOE allelic profile is at least one of APOE ε2 positive and APOE ε3 positive.

59. The device of claim 58, wherein the at least one estrogen receptor modulator includes at least one selective estrogen receptor α agonist.

60. The device of claim 58, wherein the mammalian subject's APOE allelic profile is ε4 negative.

61. The device of claim 57, wherein the mammalian subject's APOE allelic profile is ε4 positive.

62. The device of claim 61, wherein the at least one estrogen receptor modulator includes at least one selective estrogen receptor β agonist.

63. The device of claim 57, further including one or more instructions for determining the at least one treatment regimen including at least one replacement therapy for one or more steroid hormones or metabolites or modulators thereof.

64. The device of claim 63, wherein the at least one treatment regimen including the least one replacement therapy is configured to increase levels of one or more of an estrogen and a progestogen or metabolites or modulators thereof.

65. The device of claim 64, wherein the at least one treatment regimen includes replacement therapy with one or more of an estrogen and a progestogen.

66. The device of claim 57, wherein the mammalian subject is female.

67. The device of claim 66, wherein the mammalian subject is perimenopausal or postmenopausal.

68. The device of claim 66, wherein the at least one treatment regimen is determined based on the premenopausal cyclic steroid hormone levels in the mammalian subject and on current cyclic steroid hormone levels in the mammalian subject.

69. The device of claim 57, wherein the mammalian subject is male.

70.-82. (canceled)

83. A system comprising:
circuitry for determining at least one treatment regimen including at least one estrogen receptor modulator for a mammalian subject, wherein the at least one treatment regimen is determined based on the APOE allelic profile in the mammalian subject, and wherein the mammalian subject has at least one reduced steroid hormone level compared to a steroid hormone level prior to diagnosis of a cardiovascular disease or condition.

84. The system of claim 83, wherein the mammalian subject's APOE allelic profile is at least one of APOE ε2 positive and APOE ε3 positive.

85. The system of claim 84, wherein the at least one estrogen receptor modulator includes at least one selective estrogen receptor α agonist.

86. The system of claim 84, wherein the subject's APOE allelic profile is ε4 negative.

87. The system of claim 83, wherein the mammalian subject's APOE allelic profile is ε4 positive.

88. The system of claim 87, wherein the at least one estrogen receptor modulator includes at least one selective estrogen receptor β agonist.

89. The system of claim 83, further including circuitry for determining the at least one treatment regimen including at least one replacement therapy for one or more steroid hormones or metabolites or modulators thereof.

90. The system of claim 89, wherein the at least one treatment regimen including the least one replacement therapy is configured to increase levels of one or more of an estrogen and a progestogen, or metabolites or modulators thereof.

91. The system of claim 90, wherein the at least one treatment regimen includes replacement therapy with one or more of an estrogen and a progestogen.

92. The system of claim 83, further including circuitry for inputting information associated with the steroid hormone levels prior to disease diagnosis and on current steroid hormone levels in the mammalian subject.

93. The system of claim 83, wherein the at least one treatment regimen is configured to maintain a substantially physiological cyclic level of one or more steroid hormones prior to disease diagnosis in the mammalian subject in need thereof.

94. The system of claim 83, wherein the mammalian subject is female.

95. The system of claim 94, wherein the mammalian subject is perimenopausal or postmenopausal.

96. The system of claim 94, wherein the at least one treatment regimen is determined based on the premenopausal cyclic steroid hormone levels in the mammalian subject and on current cyclic steroid hormone levels in the mammalian subject.

97. The system of claim 83, wherein the mammalian subject is male.

98.-210. (canceled)

211. The system of claim 83, wherein the at least one estrogen receptor modulator is configured to maintain the mammalian subject's one or more steroid hormones or metabolites or modulators thereof at substantially physiological cyclic levels, and the at least one estrogen receptor modulator is in an amount effective to reduce cardiovascular disease or condition or alleviate symptoms thereof in the mammalian subject.

212. The system of claim 83, further including one or more instructions for inputting information associated with the steroid hormone levels prior to disease diagnosis and on current steroid hormone levels in the mammalian subject.

213. The system of claim 83, wherein the at least one treatment regimen is configured to maintain a substantially physiological cyclic level of one or more steroid hormones prior to disease diagnosis in the mammalian subject in need thereof, and the at least one treatment regimen is in an amount effective to reduce cardiovascular disease or condition or alleviate symptoms thereof in the mammalian subject.
